(19) United States
(12) Patent Application Publication
(10) Pub. No.: US 2022/0280768 A1
(43) Pub. Date: Sep. 8, 2022

(54) Cannula for Endovascular Blood Circuit Support, Corresponding Assembly, Method and Cannula System

(71) Applicant: ReCO2very Therapies GmbH, Bad Klosterlausnitz (DE)

(72) Inventors: Torsten HEILMANN, Bad Klosterlausnitz (DE); Sabine POST, Bad Klosterlausnitz (DE)

(21) Appl. No.: 17/637,579

(22) PCT Filed: Aug. 19, 2020
(86) PCT No.: PCT/EP2020/073254

§ 371 Date: Feb. 23, 2022

(30) Foreign Application Priority Data
Aug. 30, 2019 (EP) PCT/EP2019/073169

(51) Int. Cl. A61M 60/117; A61M 60/113; A61M 60/274; A61M 60/38; A61M 60/427; A61M 60/569; A61M 60/851

(52) U.S. Cl. CPC A61M 60/117; A61M 60/113; A61M 60/274; A61M 60/38; A61M 60/427; A61M 60/569; A61M 60/851

(57) ABSTRACT

Disclosed is a cannula (CA1 to CA7) for endovascular and/or jugular blood circuit support, comprising: —a proximal portion (PP1 to PP6), —a distal portion (DP1 to DP7) that comprises at least one distal opening (DO1 to DO7), —a lumen portion (LP) that extends from the proximal portion (PP1 to PP6) to the at least one distal opening (DO1 to DO7), and—at least one intermediate portion (IP1 to IP7) that is arranged between the proximal portion (PP1 to PP6) and the distal portion (DP1 to DP7), wherein the intermediate portion (IP1 to IP7) comprises at least one intermediate opening (IO1 to IO7), and wherein the intermediate portion (IP1 to IP7) is configured such that more than 90 volume percent of the fluid flow are drained from the intermediate opening (IO1 to IO7) if a fluid flow within the proximal portion (PP1 to PP6) is directed proximally and such that more than 90 volume percent of the fluid flow are delivered through the at least one distal opening (DO1 to DO7) if a fluid flow within the proximal portion (PP1 to PP6) is directed distally.

Cannula for endovascular blood circuit support, corresponding assembly, method and cannula system The invention relates to a cannula for blood circuit support that may be connected to a pump or to a variable volume reservoir such that the direction of flow, especially of blood, within a proximal portion of the cannula is alternately reversed. The cannula may have an intermediate portion comprising at least one intermediate hole, for instance with a circular or elliptical cross section or with a cross section of another shape, and/or at least one intermediate slit. Furthermore, the cannula may comprise at least one distal hole and/or at least one distal slit. The slit may have a length that is at least twice its width. The intermediate portion may allow a direction sensitive flow through the at least one intermediate hole or through the at least one distal hole depending on the direction of fluid flow within the proximal portion of the cannula.

This cannula may be used for support of the blood circuit of humans or animals, especially of the heart. A chirurgical method may be used to insert the cannula, for instance through the thorax. These chirurgical methods allow short cannulas but carry a high risk for the patient and/or may only be performed by high qualified surgeons and their teams.

It is an object of the invention to disclose a cannula for blood circuit support without chirurgical methods and/or only with minimal invasive chirurgical methods, a corresponding assembly and a corresponding method and a corresponding cannula system. The solution shall preferably reduce the risk of blood damage and/or thrombosis and/or reduce the overall health risk during insertion of the cannula into the patient. Preferably, new medical application shall be provided.

The invention is based on the consideration that the length of the cannula has to be short and that the inner diameter and therefore also the outer diameter of the cannula has to be large in order to allow high flow rates that allow special medical applications, for instance blood delivery flow rates into the body of above 4 liters per minute or above 4.5 liters per minute. Thus a cannula is proposed that avoids a major chirurgical operation because the cannula is appropriate for endovascular and/or subcutaneous insertion.

The cannula for endovascular and/or jugular blood circuit support may comprise:
a proximal portion that comprises at least one proximal opening,
a distal portion that comprises at least one distal opening,
a lumen portion or at least one lumen portion that extends from the at least one proximal opening of the cannula to the at least one distal opening of the cannula, and
at least one intermediate portion that is arranged between the proximal portion and the distal portion, preferably within the lumen portion.

The intermediate portion may be part of the lumen portion. The intermediate portion may comprise at least one intermediate opening. The at least one intermediate opening may be at least one lateral opening. In a variant a) the intermediate portion may be configured such that more than 90 volume percent of the fluid flow are drained from the intermediate opening if a fluid flow within the proximal portion is directed proximally. A pivotable flap within the intermediate portion may close the cannula in the distal direction and may open the at least one intermediate hole or opening thereby. Furthermore, the intermediate portion may be configured such that more than 90 volume percent of the fluid flow are delivered through the at least one distal opening if a fluid flow within the proximal portion is directed distally. The pivotable flap may pivot and cover the intermediate opening and may enable a flow to the distal end thereby.

In a variant b) the intermediate portion may be configured such that more than 90 volume percent of the fluid flow are drained from the at least one distal opening if a fluid flow within the proximal portion is directed proximally and such that more than 90 volume percent of the fluid flow are delivered through the intermediate opening if a fluid flow within the proximal portion is directed distally.

The cannula may be adapted to be inserted endovascularly and/or subcutaneously, e.g. a simple minimal invasive medical method may be used. Jugular insertion of the cannula allows short catheters, for instance about 65 cm (centimeter) total length of cannula, or plus and/or minus 5 percent or 10 percent of this value. Shorter length values of the cannula are possible for right jugular access, e.g. 40 cm to 60 cm, compared to longer lengths for left jugular access. Especially the right internal jugular vein and the left jugular vein may be used.

The values for the total length of the cannula may be valid for adults with at least 150 cm to 160 cm body height. The total lengths of the cannula is short if compared for instance with cannulas for femoral access.

The blood flow rates through the intermediate opening or through the distal opening may be within a range of 2.5 liters per minute to 4 liters per minute or within the range of 3 liters per minute to 3.5 liters per minute. These high flow rates may be reached based on the combination of several factors that reduce the resistance of the overall system, e.g. a short length of cannula, a great diameter, a powerful pump, etc.

The pulsatile blood delivery and drainage may have a positive effect on organ perfusion, e.g. organs have their natural conditions and are not degraded or only less degraded by the perfusion.

Access through veins may be preferred to access to arteries because there are less problems if something goes wrong, for instance tearing or disruption of a blood vessel.

The total length of the cannula may be the sum of the insertable length and of a more flexible portion that should not be inserted into the body of a subject or patient. The cannula may be reinforced along the insertable length, for instance reinforced by stiff structures, especially by wires, rings etc. The insertable length may be the length of a portion of the cannula that may be inserted into the body of a subject, e.g. into a vessel, for instance a vein or an artery. The more flexible portion may have for instance no reinforcement structures. Forceps may be used to pinch off the cannula at the flexible portion in order to interrupt the blood flow within the cannula. The more flexible portion may have a length of 5 cm plus 2.5 cm and/or minus 2.5 cm.

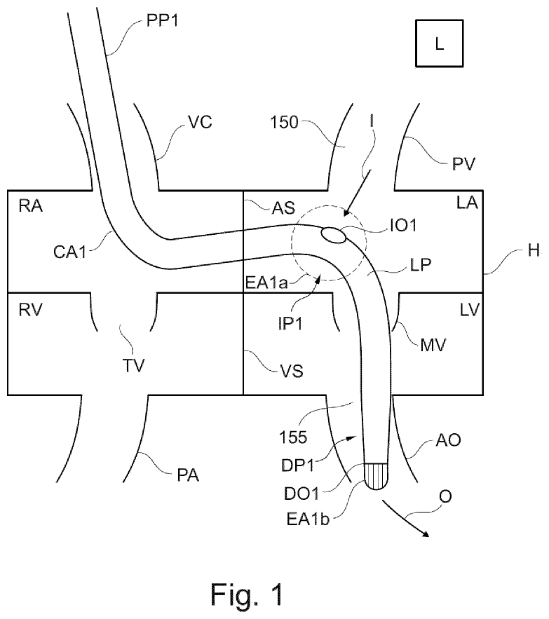

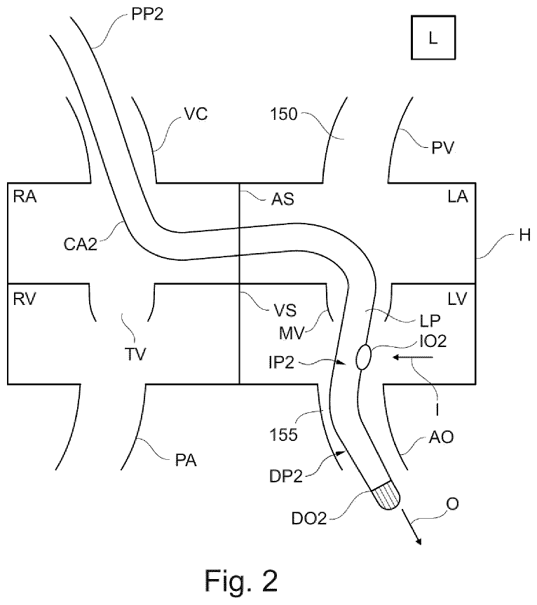

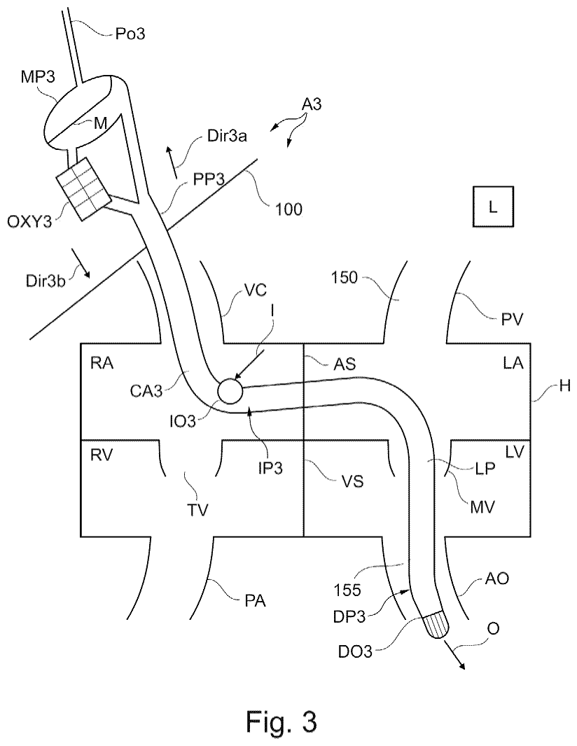

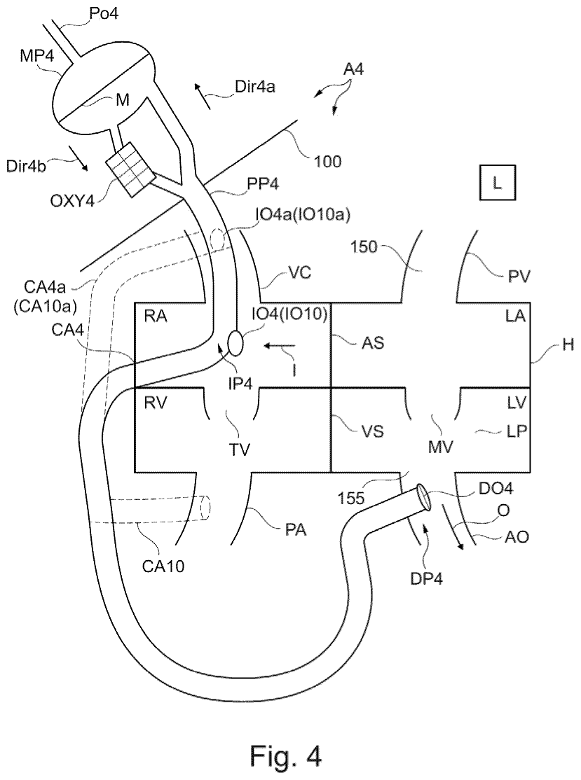

The cannula may have one of the following dimensions:
a1) Variant 1, see FIG. 1, for instance left ventricle support: A distance between a distal end of the cannula and the at least one intermediate opening may be in the range of 10 cm to 25 cm and the total length of the cannula may be in the range of 55 cm to 85 cm, preferably 65 cm. The cannula may preferably be adapted to be inserted endovascularly, preferably jugularly, through vena cava, right atrium, atrial septum, left atrium, left ventricle at least into aorta with blood drainage from the left atrium and with blood delivery into the aorta.
a2) Variant 2, see FIG. 2, for instance left ventricle support: The distance between a distal end of the cannula and the at least one intermediate opening may be in the range of 5 cm and 12 cm and a total length of the cannula may preferably be in the range of 55 cm to 85 cm, preferably 65 cm. The cannula may be adapted to be inserted endovascularly, preferably jugularly, through vena cava, right atrium, atrial septum, left atrium, left ventricle at least into aorta with blood drainage from the left ventricle and with blood delivery into the aorta.
a3) Variant 3, see FIG. 3, for instance oxygenation, e.g. lung support: A distance between a distal end of the cannula and the intermediate opening may be in the range of 22 cm to 35 cm and a total length of the cannula may be in the range of 55 cm to 85 cm, preferably 65 cm. The cannula may preferably be adapted to be inserted endovascularly, preferably jugularly, through vena cava, right atrium, atrial septum, left atrium, left ventricle at least into aorta with blood drainage from the right atrium and with blood delivery into the aorta.
a3a) Variant 3a, see FIG. 3, for instance oxygenation, e.g. lung support: A distance between a distal end of the cannula and the intermediate opening may be in the range of 27 cm to 40 cm and a total length of cannula may be in the range of 55 cm to 85 cm, preferably 65 cm The cannula may be adapted to be inserted endovascularly, preferably jugularly, through vena cava, right atrium, atrial septum, left atrium, left ventricle at least into aorta with blood drainage from the vena cava and with blood delivery into the aorta.
a4) Variant 4, see FIG. 4, for instance oxygenation, e.g. lung support: A distance between a distal end of the cannula and the intermediate opening may be in the range of 5 cm to 15 cm and a total length of the cannula may be in the range of 45 cm to 65 cm, preferably 55 cm. The cannula may preferably be adapted to be inserted endovascularly, preferably jugularly, through vena cava, right atrium and punctured transcaval from right atrium to aorta with blood drainage from the right atrium and with blood delivery into the aorta.

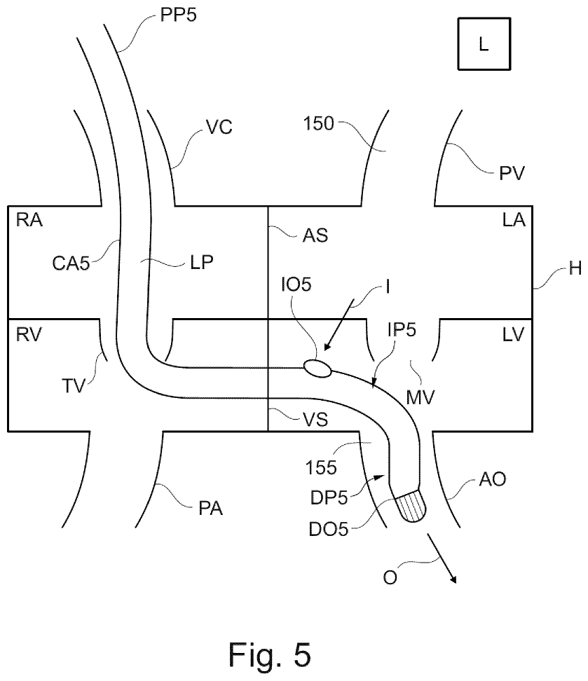

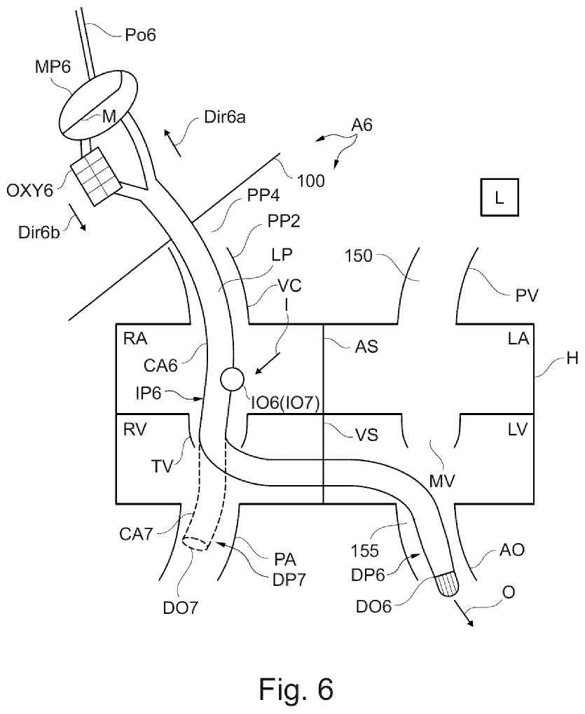

Alternatively, a distance between a distal end of the cannula and an intermediate opening may be in the range of 10 cm to 20 cm and a total length of the cannula may be in the range of 45 cm to 65 cm, preferably 55 cm. This cannula may be adapted to be inserted endovascularly, preferably jugularly, through vena cava, right atrium and punctured transcaval from right atrium to aorta with blood drainage from the vena cava and with blood delivery into the aorta.
a4a) Variant a4, see FIG. 4, for instance with oxygenation, e.g. lung support: A distance between a distal end of the cannula and the intermediate opening may be in the range of 10 cm to 25 cm and a total length of cannula may be in the range of 45 cm to 65 cm, preferably 55 cm. The cannula may be adapted to be inserted endovascularly, preferably jugular, through vena cava and punctured transcaval from the vena cava to aorta with blood drainage from the vena cava and with blood delivery into the aorta.
a5) Variant 5, see FIG. 5, for instance left heart support: A distance between a distal end of the cannula and the intermediate opening may be in the range of 10 cm to 25 cm and a total length of the cannula may be in the range of 55 cm to 85 cm, preferably 65 cm. The cannula may preferably be adapted to be inserted endovascularly, preferably jugularly, through vena cava, right atrium, right ventricle, ventricle septum, left ventricle at least to aorta with blood drainage from the left ventricle and with blood delivery into the aorta.
a6) Variant 6, see FIG. 6, for instance lung support: A distance between a distal end of the cannula and the intermediate opening may be in the range of 15 cm to 25 cm or in the range of 10 cm to 25 cm and a total length of the cannula may be in the range of 55 cm and 85 cm, preferably 65 cm. The cannula may preferably be adapted to be inserted endovascularly, preferably jugularly, through vena cava, right atrium, right ventricle, ventricle septum, left ventricle at least to aorta with blood drainage from the right atrium and with blood delivery into the aorta.

Alternatively, a distance between a distal end of the cannula and the at least one intermediate opening may be in the range of 10 cm to 20 cm and a total length of cannula is in the range of 55 cm and 85 cm, preferably 65 cm. The cannula may be adapted to be inserted endovascularly, preferably jugular, through vena cava, right atrium, right ventricle, ventricle septum, left ventricle at least to aorta with blood drainage from the right ventricle and with blood delivery into the aorta.

Further alternatively, a distance between a distal end of the cannula and the at least one intermediate opening may be in the range of 20 to 30 cm or in the range of 20 cm to 40 cm and a total length of cannula is in the range of 55 cm and 85 cm, preferably 65 cm. The cannula may be adapted to be inserted endovascularly, preferably jugular, through vena cava, right atrium, right ventricle, ventricle septum, left ventricle at least to aorta with blood drainage from vena cava and with blood delivery into the aorta.
a7) Variant 7, see FIG. 6, for instance lung support and/or right heart support: A distance between a distal end of the cannula and the intermediate opening may be in the range of 15 cm to 25 cm and a total length of cannula may be in the range of 55 cm to 85 cm, preferably 65 cm. The cannula may preferably be adapted to be inserted endovascularly, preferably jugularly, through vena cava, right atrium, right ventricle at least to pulmonary artery with blood drainage from right atrium and with blood delivery into the pulmonary artery.

Alternatively, blood drainage may be from vena cava VC and blood delivery into the pulmonary artery, e.g. the main pulmonary artery or the right pulmonary artery or the right pulmonary artery. The distance between the distal end and the intermediate opening may be increased for instance within the range of 3 cm to 5 cm in this case, see variant 9 below.
a8) Variant 8, see FIG. 6, for instance lung support and/or right heart support: The distance between a distal end of the cannula and the intermediate opening may be in the range of 10 cm and 20 cm and a total length of the cannula may be in the range of 55 cm to 85 cm, preferably 65 cm. The cannula may preferably be adapted to be inserted endovascularly, preferably jugularly, through vena cava, right atrium, right ventricle at least to pulmonary artery with blood drainage from the right ventricle and with blood delivery into pulmonary artery.
a9) Variant 9, see FIG. 6, for instance lung support and/or right heart support: A distance between a distal end of the cannula and the intermediate opening may be in the range of 25 cm to 35 cm and a total length of cannula may be in the range of 55 cm to 85 cm, preferably 65 cm. The cannula may be adapted to be inserted endovascularly, preferably jugularly, through vena cava, right atrium, right ventricle at least to pulmonary artery with blood drainage from the vena cava and with blood delivery into pulmonary artery.
a10) Variant 10, see FIG. 4, for instance lung support and/or right heart support: A distance between a distal end of the cannula and intermediate opening may be in the range of 5 cm to 15 cm and a total length of the cannula may be in the range of 45 cm to 65 cm, preferably 55 cm. The cannula may be adapted to be inserted endovascularly, preferably jugularly, through vena cava, right atrium and punctured transcaval from right atrium to the pulmonary artery with blood drainage from the right atrium and with blood delivery into the pulmonary artery.

Alternatively, a distance between a distal end of the cannula and an intermediate opening may be in the range of 10 cm to 20 cm and a total length of cannula may be in the range of 45 cm to 65 cm, preferably 55 cm. This cannula may be adapted to be inserted endovascularly, preferably jugularly, through vena cava, right atrium and punctured transcaval from the right atrium to pulmonary artery with blood drainage from vena cava and with blood delivery into the pulmonary artery.
a10a) Variant 10a, see FIG. 4: for instance lung support and/or right heart support: A distance between a distal end of the cannula and an intermediate opening may be in the range of 10 cm to 20 cm and a total length of the cannula may be in the range of 45 cm to 65 cm, preferably 55 cm. The cannula may be adapted to be inserted endovascularly, preferably jugularly, through vena cava and punctured transcaval from the vena cava to pulmonary artery with blood drainage from the vena cava and with blood delivery into the pulmonary artery.

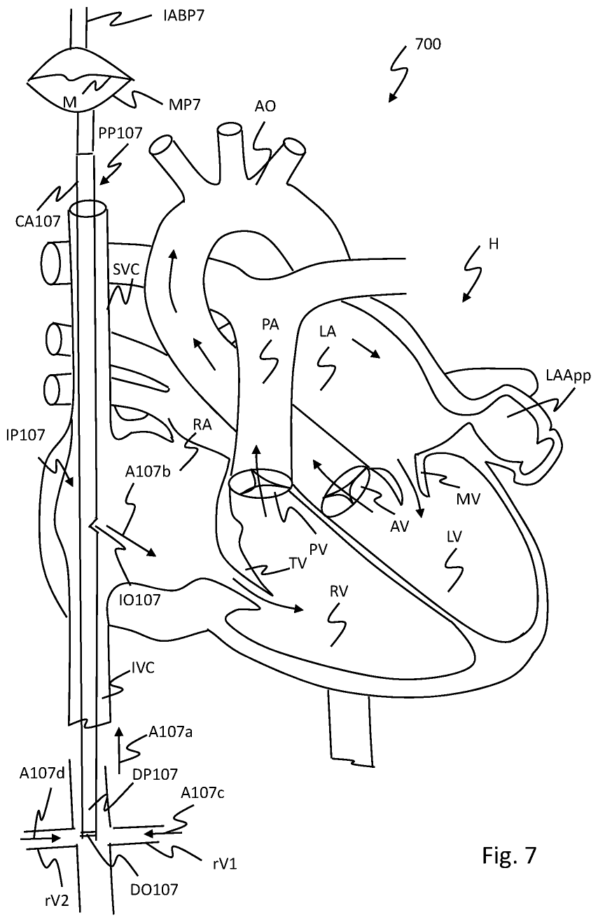

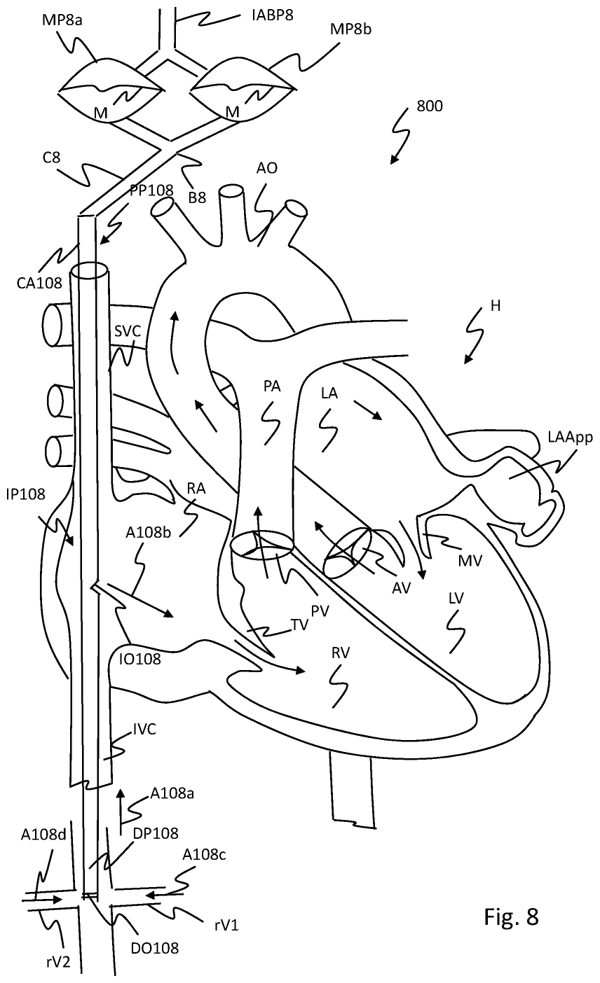

Puncturing may be made from vena cava to pulmonary artery. The drainage may be performed from vena cava in this latter case. However, the length and distance may be adapted. Alternatively the same length of the cannula as in variant 10 may be used.
a11a) Variant a11a, see FIGS. 7 and 8: a distance between a distal end of the cannula and the at least one intermediate opening may be in the range of 5 cm to 30 cm and a total length of cannula may be in the range of 35 cm to 65 cm or 45 cm to 65 cm, preferably 55 cm. The cannula may be adapted to be inserted endovascularly, preferably jugular, through superior vena cava, through right atrium and inferior vena cava with blood drainage from at least one renal vein or from both renal veins and with blood delivery into the right atrium.

In all embodiments the cannula may be inserted endovascularly through the right internal jugular vein in order to allow a short length of the cannula. The left internal jugular vein may be used alternatively, for instance because of medical reasons or because other medical devices occupy the right jugular vein.

In all embodiments, the maximal outer diameter or width of the cannula may be in the range of 25 F to 36 F or preferably in the range of 29 F to 33 F. This may allow a low fluidic resistance of the system, especially in combination with the comparably short cannulas mentioned above, i.e. appropriate for jugular access. However, greater diameters or widths may be used with care. Smaller diameters or widths of the cannulas may also be used. The cannula may have a circular cross section, an elliptical cross section or a cross section having another appropriate shape.

The same technical effects or other or additional technical effects may be valid if only the distance between the distal end of the cannula and the at least one intermediate opening are considered or if only the total length of the cannula is considered.

The cannula may comprise at least one valve for directing the fluid flows depending on the direction of the fluid flow in the proximal portion, preferably a movable and/or pivotable valve. Alternatively or additionally, the at least one valve may be arranged at the at least one intermediate opening. The at least one valve may close the intermediate opening depending on the direction of the flow in the proximal portion. It may be preferred to use only one valve because this is a simple, reliable and cost efficient solution. However, the usage of several valves may also have its advantages, for instance with regard to simplicity of the single valves.

The at least one valve may allow a direction of more than 90 percent or more than 93 percent or more than 95 percent of the volume of the flow in the main direction. At least one 1 percent of the volume of the flow and up to 4 percent or 5 percent may be allowed to flow in the secondary direction. This may allow a washout of the valve, for instance of a flap of the valve. Clotting of blood and agglutination of blood may be prevented or mitigated in this way.

The valve may comprise one of the following elements:
b1) a curved plate-shaped member that may be mounted pivotable around an axis that is arranged transversally to a longitudinal axis of the cannula, wherein the curved member may be mounted at the intermediate opening and may preferably close or open the intermediate opening. If in a non-curved state, the plate shaped member may have a circular shape or an elliptical shape.
b2) a curved plate-shaped member that may be curved along a first curvature line and that may comprise a deflector element that is curved along a second curvature line that extends within an angle of 80 to 100 degrees relative to the first curvature line, preferably with an angle of 90 degrees. The deflector element may resemble a curved pecker in a side view.
b3) a wedge shaped element, preferably comprising a first wedge shaped portion and a second wedge shaped portion, wherein preferably both wedge shaped portions point in opposite directions with regard to each other, and wherein the first wedge shaped portion has as smaller wedge angle compared to the wedge angle of the second wedge shaped portion, preferably at least 5 degrees smaller or at least 10 degrees smaller.

In all three cases b1) to b3), asymmetrical elements and/or asymmetrical arrangement of the elements may ease the change of the switch positions of the elements.

However, other kinds of valves may also be used, for instance flap valves with at least one lateral hinge or axially supported for instance along a diameter of the valve. The hinge may be for instance a film hinge made of thin plastic material. Membrane valves may also be used.

An appropriate valve material may be polycarbonate.

Alternatively, there may be no movable valve but an appropriate arrangement of openings and/or diameters of cannula for selective direction of the blood flow depending on the flow direction within the proximal portion of the cannula. Simulations of fluidic flows may be used to optimize the openings and diameters. In a further embodiment the direction sensitive fluid mechanical arrangement may be combined with at least one movable and/or pivotable valve.

The cannula may be adapted to deliver blood with a flow rate within the range of 2.5 liters per minute to 4 liters per minute or within the range of 3 liters per minute to 3.5 liters per minute. Even higher flow rates may be possible and/or may give a degree of freedom for further optimizations, for instance with regard to timing of the delivery and/or drainage of blood depending on an ECG (electrocardiography), blood pulse sensor or another sensor. The flow rate may be referred for instance to the flow rate of blood that comes out of the distal portion of the cannula. Essentially the same or the same flow rate may be drained into the cannula through the intermediate portion of the cannula.

The cannula may comprise at least one expandable arrangement at the distal portion, preferably a cage arrangement or a balloon. There may be no expandable arrangement at the intermediate portion. The expandable arrangement may have at least one, at least two, at least three arbitrarily selected or all of the following functions:
fixation of the cannula within a body, and/or
prevent “sand basting effect” during blood delivery, i.e. damage at the wall of vessels, and/or
prevent closure of inlet hole during blood drainage from to a hole of the cannula that is arranged within the body, and/or
the cage arrangement may carry a membrane for directing the fluid flow, for instance a blood flow.

The expandable arrangement may be adapted to have an expanded state and a non-expanded state. In the expanded state, a volume defined by the expandable arrangement may be greater than the volume defined by the expandable arrangement in the non-expanded state, preferably at least by factor 2, 3 or 4. The factor may be less than 100 or less than 50.

The expandable arrangement may comprise at least one inflatable balloon. There may be a separate conduit from the proximal end of the cannula to the balloon, for instance outside of the cannula or within the cannula. The balloon may be a sleeve like element that is arranged around the complete circumference of the cannula or around at least 75 percent of the circumference. The balloon may be made of a thin membrane material. A sheath member may be used during introduction of the non-inflated balloon.

Alternatively, the expandable arrangement may comprise several wires, for instance between 3 to 15 wires. A long introducer member may be used to hold the cage arrangement in its non-expanded state during insertion if the wires of the cage are connected with each other distally. A sheath member may also be used to hold the cage in its non-expanded state.

The wires may comprise a material that has a shape memory effect. The shape memory may depend on temperature or may not depend or only slightly depend on temperature. The material of the wires may comprise or consist of Nitinol (may be a registered trade mark), titanium, titanium alloys or copper-aluminum-nickel alloys. Thus, the wires may have a pre-bended shape that corresponds to the shape in the expanded state. In the non-expanded state the pre-shaped wires may be stretched for instance by an introducer member that is inserted into the expandable arrangement or by sheath member that is arranged around the expandable arrangement.

A preferred material for the wires may be a shape memory alloy (SMA) or a shape memory material, for instance a material that changes its shape depending on the temperature of the material. Nitinol (Nickel Titanium Naval Ordnance Laboratory, may be a registered trade mark) is an example for such a material.

However, other materials may also be used, for instance NiTi (nickel titan), NiTiCu (nickel titan copper), CuZn (copper zinc), CuZnAl (copper zinc aluminum) and/or CuAlNi (copper aluminum nickel). Further materials that may be used are super elastic materials, stainless steel wire, cobalt-chrome alloys or cobalt-chromium-nickel-molybdenum-iron alloys.

The thickness and/or diameter of the wires may be in the range of 0.1 mm (millimeter) to 2 mm, especially if only three or four wires are used within the expandable arrangement that may also be named as a cage arrangement. The thickness and/or diameter of the wires may be in the range of 0.1 mm (millimeter) to 1 mm or in the range of 0.25 mm to 0.75 mm. Thinner wires may be useful if more than four wires are comprised within the cage arrangement.

The cannula may comprise at least one expandable arrangement at the intermediate portion, preferably a cage arrangement or a balloon. The same features as mentioned above for an expandable arrangement at the distal portion may apply. For instance, a sheath member may be used to hold the cage arrangement in non-expanded state during insertion. There may be an expandable arrangement at the intermediate portion but not on the distal portion.

The cannula may comprise at least one first expandable arrangement at the distal portion, preferably a first cage arrangement or a first balloon, and at least one second expandable arrangement at the intermediate portion, preferably a cage arrangement or a balloon. Only balloons may be used at one cannula. Alternatively, only cage arrangements may be used at one cannula. However, a combination of a cage arrangement and a balloon is possible as well, for instance a distal balloon and a cage arrangement at the intermediate portion, e.g. around the intermediate opening. The cage arrangement may not block a vessel or chamber as compared to a balloon that may be designed to seal a vessel. However, a balloon may be designed to not block a vessel but to provide for instance a fixation.

The wall thickness of the cannula may be within the range of 0.1 mm to 0.5 mm. This range may allow larger inner diameters or inner widths compared to thicker wall thicknesses for the same maximal outer diameter. Independent of the wall thickness, the cannula may have constant inner diameters and/or outer diameters along its complete insertable length or along at least 75 percent of the insertable length. Alternatively, the cannula may have decreasing inner diameters and/or outer diameters along its complete insertable length or along at least 75 percent of the insertable length. This may ease the insertion of the cannula but may reduce the flow rates to some degree.

The wall of the cannula may be reinforced by wires, especially by metal wires, or by plastic fibers or by glass fibers.

The inner wall of the cannula may carry at least one structure that effects a rotation of the fluid flow within the cannula. The structure may be helically wound and/or comprise protrusions or recesses. The rotation of the fluid flow may stabilize the flow, e.g. prevent turbulences. Laminar flows may be promoted by the structure that effects a rotation of the fluid flow within the cannula.

A further aspect of the invention relates to an assembly or set/kit for endovascular blood circuit support, comprising:
at least one cannula according to one of the embodiments mentioned above, and
at least one variable volume reservoir that has an aspiration phase or an aspiration operating phase for drawing fluid into the variable volume reservoir (but out of the cannula) and that has an expulsion phase or an expulsion operating phase for pressing the fluid out of the variable volume reservoir (e.g. delivery blood into the cannula), or
alternatively, a pump that may be controlled to drive a fluid flow within the cannula into two different directions.

The cannula is coupled or may be adapted to be coupled directly to the at least one variable volume reservoir or to the pump. Alternatively, the assembly may comprise at least one coupling conduit that is coupled or that is adapted to be fluidically coupled between the at least one cannula and the at least one variable volume reservoir or pump.

The variable volume reservoir may comprise a casing and a flexible membrane within the casing. Alternatively other types of membrane pumps, a piston arrangement, a bellow etc. may be used. The advantage may be that there may be no rotating parts that are in contact with the blood of a subject. No shear stress or only low shear stress may be impacted to blood molecules. This may result in no damage or only less damage of blood molecules. Thus these molecules may fulfill their complex natural function further, e.g. oxygen transport, immune functions, etc.

The variable volume reservoir may be a membrane pump that is for instance operated with helium or other gaseous fluids. Temperature control may be used in order to prevent that the temperature of the blood rises above or falls below normal blood temperature.

However, other pumping devices may also be used, for instance a centrifugal pump an axial pump or a diagonal pump. These pumps may allow higher flow rates compared to the usage of a variable volume reservoir.

The variable volume reservoir or the pump may form separate devices that may be coupled with each other to form a fluid circuit. This allows higher flexibility. The cannula and/or reservoir may be disposable devices. Furthermore, it is possible to easily insert the cannula first using for instance an introducer member and/or a guide wire. After the insertion of the cannula the introducer member and/or the guide wire may be removed and the cannula may be coupled to the pump or to the variable volume reservoir.

The variable volume reservoir may comprise at least one membrane, preferably a flat membrane or a toroidal membrane. The membrane may be made of polycarbonate, poly(methyl methacrylate) PMMA, silicone, or of another appropriate material.

A piston pump arrangement may be used to control the variable volume reservoir.

Alternatively, the variable volume reservoir may be formed by a piston arrangement.

However, the usage of a membrane opens the possibility for a good temperature control of the blood.

The inflation/deflation frequency may be in the range of 60 to 90 times per minute or in the range of 70 to 80 times per minute. Thus, every heart beat may be used to deliver blood into the blood circuit of subject.

Alternatively, it may be advantageously to deliver blood only every second heartbeat or every third heartbeat for instance in order to improve timing based for instance on ECG (electrocardiography) data or signals or on other signal. A timing rate of 50/50 may be used for pumping blood into the body and out of the body. However, other timing rates are also possible, for instance more time for pumping blood into the body and less time for pumping blood out of the body or vice versa. The difference may be at least 10 percent or twenty percent of the greater value. There may be no pause between the switching of the direction of blood flow in the proximal part of the cannula, i.e. no pause that is longer as a minimum that is technically necessary for switching. Alternatively, there may be a pause or a longer pause between the switching from drainage to delivery and vice versa.

The displacement device, e.g. variable volume reservoir, or the pump may be arranged near the body of a subject to allow short fluidic circuitries. The distance between the entry point of the cannula into the body and the variable volume reservoir/pump may be in the range of 5 cm to 15 cm and may be less than for instance 20 cm.

The pressures that may be generated by the variable volume reservoir/pump may be within the range of 300 mmHg (mm (millimeter) mercury (quicksilver) column) or 400 mmHg to 600 mmHg. This range is appropriate to prevent damage at all or to prevent severe damage of blood cells.

The variable volume reservoir may comprise two ports for blood transfer, preferably at the same side of the membrane or of a membrane. The ports may be used to establish a circular blood flow through the variable volume reservoir. Both ports may be connected to the cannula using for instance a Y-connector or a T-connector having a bifurcation and three ports that are connected with each other. Each port of the variable volume reservoir may comprise or may be associated with a valve, for instance with a one-way valve respectively. Alternatively the valves may be comprised within the connector or at other appropriate places within the circuitry. The valve and/or other directional sensitive arrangement within the intermediate portion of the cannula may be used further in order to maintain the overall function of the fluidic circuitry. Two ports of the variable volume reservoir may be useful to include devices that are optimized for a one-directional flow into a circuitry that has a portion with bidirectional flow, e.g. the proximal portion of the cannula. An oxygenator device and/or a filter unit and/or a drug delivery device may be such one-directional device, preferably an extracorporeal device.

Alternatively, the variable volume reservoir may comprise only one port that is connected with the cannula, i.e. only one port for blood transport. This may simplify the variable volume reservoir and the overall fluidic circuitry. Furthermore, other medical devices may be included if they are operable or optimized for a bi-directional flow, for instance an oxygenator and/or a filter unit and/or a drug delivery device, preferably an extracorporeal device.

An oxygenator device may be used to raise the blood level of the subject to a normal blood level or to a higher blood level than normal. This may support the lung function of a subject. Alternatively or additionally, a carbon dioxide removal device may be used to support the lung.

The oxygenator device may be adapted to be inserted or is inserted fluidically within one secondary branch of a fluid circuit only. The fluid flow may flow through the oxygenator only in one direction. This may allow the usage of a commercially available oxygenator or of an oxygenator device that has a comparably simple construction.

Alternatively, the oxygenator device may be adapted to be inserted or is inserted into a main branch of a fluid circuit between the cannula and the variable volume reservoir. The fluid flow may flow through the oxygenator device in two directions, for instance to improve washout of the oxygenator device. The circuitry may remain simple in this case, i.e. only one main line for blood transfer and no bifurcation elements except for instance within the intermediate portion of the cannula.

The variable volume reservoir may be adapted to be used with an IABP (Intra-Aortic Balloon Pump) console that is not part of the assembly. Alternatively, the assembly may comprise a control unit that is able to control the variable volume reservoir or the pump depending on the heartbeat and/or on pulse beat that is measured by at least one sensor, for instance a known IABP (Intra-Aortic Balloon Pump) or another control unit. IABP (Intra-Aortic Balloon Pump) devices are widely used for other purposes in many clinics and hospitals. Thus, there may be no extra costs involved for these control units. The control unit may receive ECG (electrocardiography) signal or other signals or data that allow control of the variable volume reservoir or of an equivalent pump.

The variable volume reservoir may have a maximal pump volume that is equal to or greater than 50 ml (milliliter) or equal to or greater of 60 ml, preferably within the range of 60 ml to 160 ml or most preferably within the range of 80 ml to 120 ml. This volume may refer to the difference of the volumes between the expulsion phase and the aspiration phase. A higher volume may allow a higher pumping rate.

The volume may be appropriately selected with regard to the volume of the lumen portion of the cannula/catheter and/or a conduit between the cannula and an input port of the variable volume reservoir.

Preferably, the variable volume of the variable volume reservoir may be greater than the sum of the volume of the lumen portion of the cannula and the volume of the conduit. The variable volume of the variable volume reservoir may be for instance within the range of plus 5 percent to 30 percent of the sum of the volumes of the lumen portion and of the conduit. This may result in low or no blood clotting. Good oxygenation may be reached if an oxygenator is used. No dead ends may be generated within the circuitry if both volumes are selected appropriately.

Alternatively, an equivalent other pump may be used as mentioned above. Again it may be advantageous to make sure that the whole blood volume within the circuitry is changed completely or almost completely (for instance more than 90 percent of volume) during each cycle of the bi-directional operation of the pump.

A third aspect of the invention relates to a method for endovascular blood circuit support. The method may comprise:
inserting a cannula endovascularly through a vessel of the blood circuit, and
drawing blood mainly from the at least one intermediate opening during a drawing phase, preferably during an aspiration phase of a variable volume reservoir, and delivering blood out of the at least one distal opening during a delivery phase, preferably an expulsion phase of the variable volume reservoir.

Instead of the variable volume reservoir a pump may be used to realize the drawing phase and the delivery phase.

The cannula may be a cannula according to one of the embodiments mentioned above. The cannula may be preferably inserted jugularly. A jugular vein may be preferred for insertion instead of a subclavian artery. Injured veins may be repaired easier than for instance injured arteries because of the lower blood pressure within veins compared to the blood pressure in arteries. Thus, it may be easier to stop bleeding out of a vein than out of an artery. The jugular access allows short cannulas resulting in a smaller fluidic resistance. The smaller fluidic resistance may enable higher flow rates and/or higher dynamics of blood drainage out of and/or blood delivery into the body of a subject or patient. The advantages that are mentioned above for the cannula also apply to the method.

Variant 1 and 2, see FIGS. 1 and 3: The distal portion of the cannula may be inserted endovascularly, preferably jugularly, through vena cava, right atrium, atrial septum, left atrium, left ventricle at least to ascending aorta. Blood may be drained into the at least one intermediate opening from the left atrium or blood may be drained into the at least one intermediate opening from the left ventricle and blood may be delivered out of the at least one distal opening into the aorta. This may be done without oxygenation.

However, additionally oxygenation may be possible to support not only the heart but also the lung. The length of the cannula and/or the distance between the distal end and the intermediate opening may be selected as mentioned above, i.e. for variant 1 or 2.

Variant 3 and 3a, FIG. 3: The distal portion of the cannula may be inserted endovascularly, preferably jugularly, through vena cava, right atrium, atrial septum, left atrium, left ventricle at least to ascending aorta. Blood may be drained into the at least one intermediate opening from the right atrium or from the vena cava. Blood may be delivered out of the at least one distal opening into the aorta. The blood may be oxygenated after it is drained in and before it is delivered out, preferably by at least one extracorporeal oxygenator, e.g. an oxygenator that has not to be implanted and/or that has a large oxygenation power.

The length of the cannula and/or the distance between the distal end and the intermediate opening may be selected as mentioned above, i.e. for variant 3 or 3a.

Transcaval access may be used for instance if valves of the heart have a disease that prevents the insertion of a cannula through these valves. There may be at least four variants a) to d) of the method for a transcaval access:
a) Variant 4a, FIG. 4, transcaval from VC to AO with oxy: The distal portion of the cannula is inserted endovascularly, preferably jugularly, through vena cava and punctured from the vena cava directly to aorta. Blood may be drained into the at least one intermediate opening from the vena cava and blood may be delivered out of the at least one distal opening into the aorta. Blood may be oxygenated after it is drained in and before it is delivered out of the cannula, preferably by at least one extracorporeal oxygenator. The length of the cannula may be selected as mentioned above for variant 4a. The distance between the distal end and the intermediate opening may be selected as mentioned above for variant 4a.
b) Variant 4, FIG. 4, transcaval from right atrium to aorta with oxygenator: The distal portion of the cannula may be inserted endovascularly, preferably jugularly, through vena cava, right atrium and punctured from the right atrium directly to aorta. Blood may be drained into the at least one intermediate opening from the vena cava or from the right atrium and wherein blood may be delivered out of the at least one distal opening into the aorta. The blood may be oxygenated after it is drained in and before it is delivered out, preferably by at least one extracorporeal oxygenator. The length of the cannula and/or the distance between the distal end and the intermediate opening may be selected as mentioned above for variant 4.
c) Variant 10a, FIG. 4, transcaval from vena cava to pulmonary artery, for instance for a right ventricle assist device (RVAD), preferably without oxygenation or with oxygenation, depending on what the patient needs: The distal portion of the cannula may be inserted endovascularly, preferably jugularly, through vena cava and punctured from the vena cava directly to a pulmonary artery, for instance into the main pulmonary artery or into the left or right pulmonary artery. Blood may be drained into the at least one intermediate opening from the vena cava and blood may be delivered out of the at least one distal opening into the pulmonary artery. The distance between the distal end and the intermediate opening may selected as mentioned above for variant 10a. The total length of the cannula may be the same as mentioned above for variant 10a, i.e. for instance in the range of 45 cm (centimeter) to 65 cm, preferably 55 cm.
d) Variant 10, FIG. 4, transcaval from right atrium to pulmonary artery, for instance for a right ventricle assist device (RVAD), preferably without oxygenation or with oxygenation, depending on what patient needs: The distal portion of the cannula may be inserted endovascularly, preferably jugularly, through vena cava, right atrium and punctured from the right atrium directly to a pulmonary artery, for instance into the main pulmonary artery or into the left or right pulmonary artery. Blood may be drained into the at least one intermediate opening from the vena cava or from the right atrium and blood may be delivered out of the at least one distal opening into the pulmonary artery. The length of the cannula and/or the distance between the distal end and the intermediate opening may be selected as mentioned above for variant 104, i.e. transcaval to pulmonary artery.

Variant 5, FIG. 5, for instance left ventricle assist device (LVAD): The distal portion of the cannula may be inserted endovascularly, preferably jugularly, through vena cava, right atrium, right ventricle, ventricle septum, left ventricle at least to ascending aorta. Blood may be drained into the at least one intermediate opening from the left ventricle and blood may delivered out of the at least one distal opening into the aorta. The length of the cannula and/or the distance between the distal end and the intermediate opening may be selected as mentioned above for variant 5. Oxygenation may be performed or not depending on patients need for lung support.

Variant 6, FIG. 6, for instance lung support: The distal portion of the cannula may be inserted endovascularly, preferably jugularly, through vena cava, right atrium, right ventricle, ventricle septum, left ventricle at least to ascending aorta. Blood may be drained into the at least one intermediate opening from the vena cava or from the right atrium or from the right ventricle and blood may be delivered out of the at least one distal opening into the aorta. The blood may be oxygenated after it is drained in and before it is delivered out of the cannula, preferably by at least one extracorporeal oxygenator. The length of the cannula and/or the distance between the distal end and the intermediate opening may be selected as mentioned above for variant 6.

The cannula may be inserted into the internal jugular vein. The right internal jugular vein may allow the usage of shorter cannulas. However, usage of the left internal jugular vein is also possible, for instance if the right internal jugular vein is used for another cannula or if there are medical reasons why the right internal jugular vein should not be used. This is true for all variants 1 to 10 and even for variant 11 and for all sub variants and embodiments mentioned above and below if not stated otherwise.

The lower half of the ranges given for the length of the cannula may be valid for access through the right jugular vein. The upper half of the ranges given for the length of the cannula may be valid for access through the left jugular vein. Exemplary, the range of 55 cm to 85 cm may have a lower half from 55 cm to 70 cm and an upper half from 70 cm to 85 cm.

Variant 7, 8 and 9, FIG. 6, for instance right ventricle assisted device (RVAD), preferably without oxygenation or with oxygenation, depending on what patient needs: The distal portion of the cannula may be inserted endovascularly, preferably jugularly, through vena cava, right atrium, the right ventricle at least to the main pulmonary artery. Blood may be drained into the at least one intermediate opening from the vena cava or from the right atrium or from the right ventricle and blood may delivered out of the at least one distal opening into the pulmonary artery.

The total length of the cannula and the distance between distal tip and intermediate opening for draining from vena cava may be as follows:
distance is in the range of 25 cm to 35 cm, and/or
total length of cannula in the range of 55 cm to 85 cm, preferably 65 cm, i.e. as mentioned above under item a9), i.e. for variant 9.

The total length of the cannula and the distance between the distal tip and the intermediate opening for draining from right atrium may be as follows:
distance in the range of 15 cm to 25 cm, and/or
total length of cannula in the range of 55 cm to 85 cm, preferably 65 cm, i.e. as mentioned above under item a7), i.e. for variant 7.

The total length of the cannula and the distance between the distal tip and the intermediate opening for draining from right ventricle may be as follows:
distance in the range of 10 cm to 20 cm, and/or
total length of cannula in the range of 55 cm to 85 cm, preferably 65 cm, i.e. as mentioned above under item a8), i.e. for variant 8.

Variant 11: A fourth aspect that may be claimed later, for instance in a divisional application, relates to a method for endovascular blood circuit support. The method may comprise:
inserting a cannula endovascularly through a vessel of the blood circuit, preferably a cannula according to an embodiment that is mentioned above, for instance comprising an expandable arrangement,
wherein the cannula comprises:
a proximal portion that comprises at least one proximal opening,
a distal portion that comprises at least one distal opening,
at least one lumen portion that extends from the at least one proximal opening of the cannula to the at least one distal opening of the cannula, and
at least one intermediate portion that is arranged between the proximal portion and the distal portion, and wherein the intermediate portion comprises at least one intermediate opening, preferably at least one lateral opening,
wherein the intermediate portion is configured such that more than 90 volume percent of the fluid flow are drained through the distal opening if a fluid flow within the proximal portion is directed proximally and such that more than 90 volume percent are delivered through the at least one intermediate opening if a fluid flow within the proximal portion is directed distally,
draining blood mainly from the distal opening through the intermediate portion into the proximal portion during a drainage phase, for instance an aspiration phase of a variable volume reservoir, and delivering blood out of the at least one intermediate opening during a delivery phase, for instance an expulsion phase of the variable volume reservoir.

The intermediate portion may be part of the lumen portion. New medical applications may be opened up by this method, especially if the cannula is punctured through a septum of the heart, e.g. through the atrial septum or through the ventricle septum. Furthermore, new medical applications may be opened up if the cannula is used transcaval.

The distal portion of the cannula according to the fourth aspect may be inserted endovascularly, preferably jugularly, through aorta, preferably ascending aorta, into the left ventricle. Blood may be drained into the at least one distal opening from the left ventricle and blood may be delivered out of the at least one intermediate opening into the aorta.

For the methods according to the third aspect and according to the fourth aspect, the proximal portion of the cannula may be coupled to a variable volume reservoir that may perform the aspiration phase for drawing fluid into the reservoir and that may perform the expulsion phase for pressing the fluid out of the reservoir or to a pump. The variable volume reservoir may be an extracorporeal reservoir that does not need miniaturization and/or implantation. The pump may be a pump that allows pulsatile operation, preferably an extracorporeal pump.

The pump may be a pump that may also be operated in a continuous mode. However, the pump may be operated in a pulsatile mode by fast accelerating a rotor in a first rotation direction, then stopping the rotor, and thereafter fast accelerating the rotor in a second rotation direction that is opposite to the first rotation direction. This may be repeated in a cyclic manner.

A control unit may be used that is able to control the variable volume reservoir or the pump depending on the heartbeat and/or on pulse beat that is measured by at least one sensor. The control unit may be used for the methods according to the third aspect or the fourth aspect and their embodiments.

The control unit may control the variable volume reservoir or the pump such that every heartbeat, preferably of the left ventricle, blood is delivered into a body of a subject. This may allow high flow rates of blood delivery and/or blood drainage.

Alternatively, the control unit may control the variable volume reservoir or the pump such that every second heartbeat, preferably of the left ventricle blood is delivered into a body of a subject. This means that at least one heart beat is skipped. More time may be available for exact timing to be synchronous with the heartbeat. The average flow rate may be lowered. However, high maximum flow rates may still be used.

The maximum flow rate may be in the range of 2.5 liters per minute to 4 liters per minute or within the range of 3 liters per minute to 3.5 liters per minute divided by two for instance. The flow rates may be divided by two if only every second heartbeat is used for blood delivery. However, higher flow rates or lower flow rates may also be used.

The switchable control unit may have several modes, for instance a first mode in which every heart beat is used for blood delivery and blood drainage and a second mode in which only every second heartbeat or other interval is used for blood delivery out of the cannula.

The cannula may be introduced or inserted endovascularly, preferably jugular, through a septum of the heart. This may allow new medical application scenarios for the bi-directional cannula, i.e. a cannula that is used with a bi-directional flow in its proximal part. Some of these scenarios are mentioned above and/or described in more detail in the Figures. However, many more scenarios and application possibilities may be found.

The cannula may be punctured and/or inserted through the atrial septum. The atrial septum may be easier to reach endovascularly compared to the ventricle septum. There may be medical reasons to use the atrial septum.

Alternatively, the cannula may be punctured and/or inserted through the ventricle septum because it is more appropriate than the atrial septum. The ventricle septum may be used if the atrial septum may not be used, for instance because of medical reasons. The atrial septum may have a disease or may be punctured too often. Moreover, medical devices may occupy the atrial septum.

The cannula may be introduced or inserted endovascularly, preferably jugularly, through the vena cava. The cannula may be punctured transcaval from vena cava or from right atrium at least to the aorta or to the aorta or into a pulmonary artery, for instance into the main pulmonary artery or into the right pulmonary artery. This opens room for new medical applications and scenarios for the bi-directional cannula, i.e. a cannula that is used with a bi-directional flow in its proximal part. Some of these scenarios are mentioned above and/or described in more detail in the Figures. However, many more scenarios and application possibilities may be found. The transcaval way avoids a passage through the heart or through more than one chamber of the heart. Thus, the heart may pulse without disturbance through the cannula.

For all disclosed method embodiments, the cannula may have a maximal outer diameter in the range of 25 Fr (French, 1 French equals to ⅓ millimeter) to 36 Fr or, preferably, in the range of 29 Fr to 33 Fr. 33 Fr are still usable for insertion through a jugular vein. Thus, it is possible to deliver and/or drain high flow rates of blood or to have a further degree of freedom if lower flow rates are necessary than theoretically and/or practically possible. The degree of freedom may be used for instance for improving the timing of the blood delivery and/or drainage from accordance with an ECG (electrocardiography).

The cannula or embodiments of the cannula, and/or the assembly and the embodiments of the assembly may be used to perform the method or its embodiments mentioned above or below. Thus corresponding technical effects may apply. Vice versa, the cannula or the assembly and their embodiments may have features which are mentioned only for the methods or their embodiments mentioned above or below. These features may also be used for the devices and may have the same or similar technical effects.

The distal portion of the bidirectional cannula may be inserted endovascularly, preferably jugularly, through superior vena cava, right atrium and inferior vena cava at least to or to a location which may have a distance to the junction of the renal veins into the inferior vena cava equal to 10 cm (centimeter) or less than 10 cm, equal to 5 cm or less than 5 cm or equal to 2.5 cm or less than 2.5 cm. Blood may be drained into the at least one distal opening, preferably from the junction, and blood may be delivered out of the at least one intermediate opening into the right atrium. Thus, blood may be pulled from the kidneys, for instance to stimulate the function of the kidney(s).

The cannula may be connected with only one membrane pump or with at least two membrane pumps which are preferably operated in a parallel operation mode.

The cannula may be a bidirectional cannula and the bidirectional cannula may be inserted through at least one vessel of the blood circuit within an outer cannula which has been already arranged in the at least one vessel of the blood circuit. The outer cannula may comprise:
a proximal portion,
a distal portion that comprises at least one distal opening,
a lumen (portion) that extends from the proximal portion to the at least one distal opening,
at least one intermediate portion that is arranged between the proximal portion and the distal portion, and
in the intermediate portion of the outer cannula, at least one intermediate opening, preferably a lateral opening, which is configured to allow passage of the distal portion of the bidirectional cannula. New medical applications may be possible using a cannula system comprising the bidirectional cannula and the outer cannula, for instance for treatment and/or support of the heart and/or lung and/or kidney(s) or other organs.

The outer cannula may be inserted through the at least one vessel of the blood circuit before inserting the bidirectional cannula. The bidirectional cannula may be inserted into the outer cannula until the distal portion of the bidirectional cannula extends through the lateral intermediate opening of the outer cannula and/or the intermediate opening of the bidirectional cannula may be arranged within the intermediate portion of the bidirectional cannula. Thus, the outer cannula guides the bidirectional cannula as long as the bidirectional cannula is within the outer cannula.

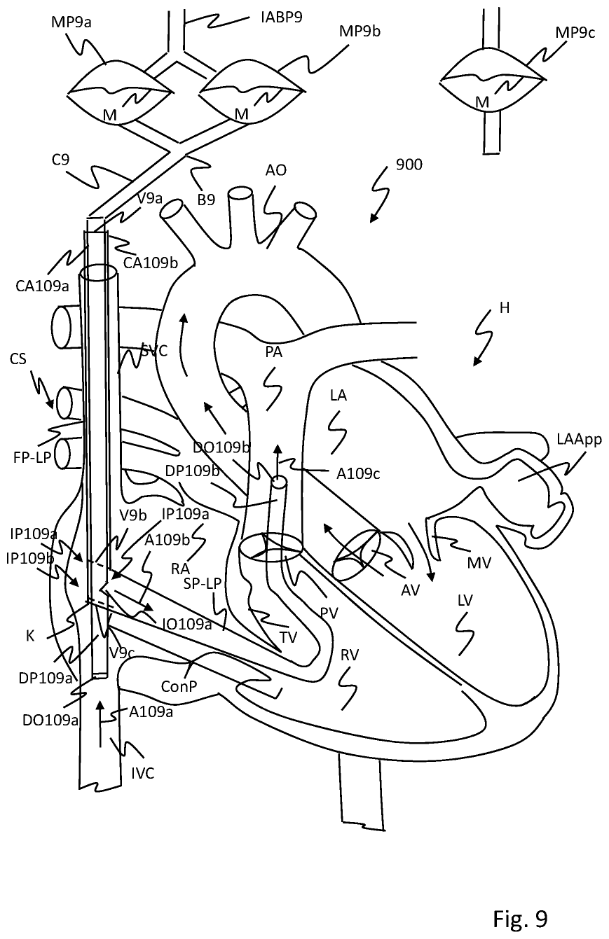

The distal portion of the outer cannula may be inserted endovascularly, preferably jugularly, through superior vena cava, right atrium, right ventricle at least to the pulmonary artery. The distal portion of the bidirectional cannula may be inserted into the right atrium or into the inferior vena cava. Blood may be drained into the at least one distal opening of the bidirectional cannula. Blood may be delivered out of the at least one intermediate opening of the bidirectional cannula and further through the at least one distal opening of the outer cannula. Thus, right heart support (see for instance FIG. 9) may be established in a simple manner.

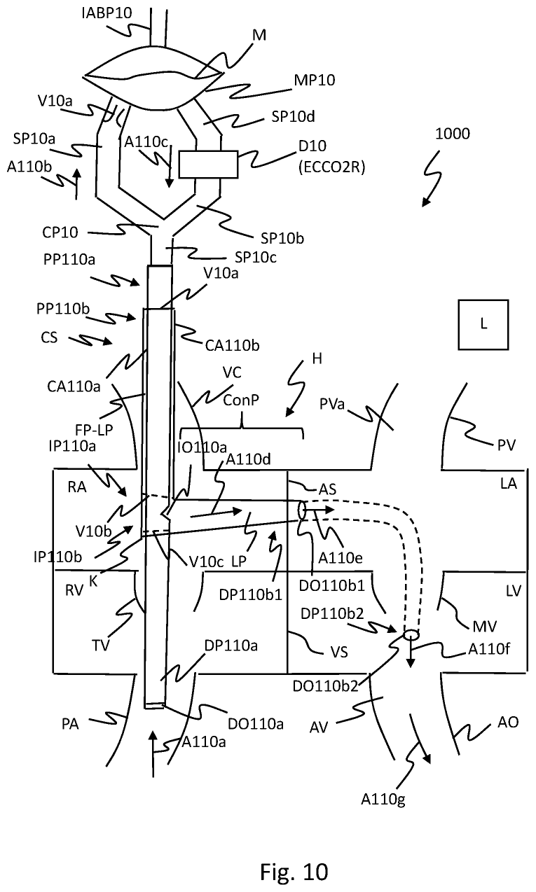

The distal portion of the outer cannula may be inserted endovascularly, preferably jugularly, through the superior vena cava, the right atrium and the atrial septum to the left atrium of the heart or to the left ventricle or at least to the left ventricle. The distal portion of the bidirectional cannula may be inserted into the right atrium, through the right ventricle and at least to the pulmonary artery. Blood may be drained into the at least one distal opening of the bidirectional cannula. Blood may be delivered out of the at least one intermediate opening of the bidirectional cannula and further through the at least one distal opening of the outer cannula. Thus, for instance an ECCO₂R (extracorporeal carbon dioxide removal) may be established in a simple manner (see for instance FIG. 10).

The drained blood may be enriched with oxygen and/or depleted from carbon dioxide outside of the body of a patient before it is delivered out of the at least one intermediate opening of the bidirectional cannula, especially if the cannula system is used.

A further aspect relates to a cannula system, comprising:
a bidirectional cannula according to one of the embodiments mentioned above, and
an outer cannula. The outer cannula may comprise:
a proximal portion,
a distal portion that comprises at least one distal opening,
a lumen or a lumen portion that extends from the proximal portion to the at least one distal opening,
at least one intermediate portion that is arranged between the proximal portion and the distal portion, and
in the intermediate portion of the outer cannula at least one intermediate opening, preferably a lateral opening, which is configured to allow passage of the distal portion of the bidirectional cannula.

The bidirectional cannula and the outer cannula may be configured such that when the bidirectional cannula is inserted into the outer cannula, the distal portion of the bidirectional cannula extends through the lateral intermediate opening of the outer cannula. In the inserted state, the intermediate opening of the bidirectional cannula may be arranged within the intermediated portion of the outer cannula fluidly connected to the distal portion of the outer cannula.

The bidirectional cannula and the outer cannula may be configured such that when the bidirectional cannula is inserted into the outer cannula, a further lumen portion may be defined between an outer surface of the bidirectional cannula and an inner surface of the outer cannula. The further lumen portion may be closed at its distal end and/or at its proximal end. Thus, the lumen between the two cannulas may be a “dead” lumen which is not used for blood transport. Valves or other sealing elements at the end(s) of the further lumen portion may prevent that blood flows into the dead lumen.

The outer diameter of the bidirectional cannula may be at most 4 French (1 French equal to ⅓ mm (millimeter)) or at most 2 French smaller than the outer diameter of the outer cannula, preferably in a portion along the length or along the longitudinal axis of the bidirectional cannula between the proximal portion of the bidirectional cannula and the intermediate portion of the bidirectional cannula when the bidirectional cannula is inserted into the outer cannula. Thus, the “dead” lumen may be as small as possible. Each cannula may have an inner diameter as large as possible in order to enable high blood flow rates through the bidirectional cannula and through the outer cannula.

The cannula system may comprise a proximal valve, preferably a hemostasis valve, at the proximal portion of the outer cannula. The proximal valve may be configured to allow insertion of the bidirectional cannula through the proximal hemostatic valve into the outer cannula. The proximal valve may prevent that blood flows out of the outer cannula or into the outer cannula as long as the outer cannula is inserted into the body but the bidirectional cannula is not yet inserted into the outer cannula.

Alternatively or additionally, the cannula system may comprise an inner intermediate valve, preferably a hemostasis valve, at the intermediate portion of the outer cannula. The intermediate valve may be configured to allow insertion of the bidirectional cannula through the intermediate hemostatic valve. The inner intermediate valve may prevent that blood flows into the “dead” lumen.

Further, alternatively or additionally, the cannula system may comprise a valve, preferably a hemostasis valve, at the lateral intermediate opening of the outer cannula. The intermediate valve at the intermediate opening may be configured to allow passage of the distal portion of the bidirectional cannula through the intermediate hemostatic valve. The valve at the intermediate opening of the outer cannula may prevent undesired blood flows during usage of the cannulas.

The outer cannula may comprise a kink in the intermediate portion of the outer cannula, preferably in a base state in which no outer forces are applied to the outer cannula disregarding gravity. The kink may include an angle in the range of 80 degrees to 130 degrees, preferably 110 degrees. The kink may simplify insertion of the outer cannula and/or it may facilitate the insertion of a further cannula, e.g. an inner cannula. The inner cannula may be a bidirectional cannula or a cannula for unidirectional flow only.

The intermediate opening of the outer cannula may be arranged at the kink. This may alleviate the insertion of the bidirectional cannula through the intermediate opening of the outer cannula.

The cannula system may be adapted to be used for the method and its embodiments mentioned above. Thus, the same technical effects may apply.

With regard to the assembly as mentioned above, the at least one cannula may be a bidirectional cannula. The assembly may further comprise an outer cannula. The bidirectional cannula may be adapted to be inserted into the outer cannula. The outer cannula may comprise the portions and openings that are mentioned above.

Alternatively, a kit may be protected which comprises the parts of the assembly loosely.

The assembly and/or the kit may further comprise at least two variable volume reservoirs.

A further aspect relates to a cannula, preferably to the outer cannula of a cannula system as mentioned above or to an outer cannula of an assembly as mentioned above. The cannula may comprise the same parts that are mentioned above for the outer cannula, e.g.
a proximal portion,
a distal portion that comprises at least one distal opening,
a lumen or a lumen portion that extends from the proximal portion to the at least one distal opening, and
at least one intermediate portion that is arranged between the proximal portion and the distal portion, wherein the intermediate portion of the outer cannula comprises at least one intermediate opening, preferably a lateral opening, which is configured to allow passage of the distal portion of a further cannula, preferably of a bidirectional cannula.

The outer cannula may be an essential part of the cannula system mentioned above. Thus, the same technical effects may apply. Furthermore, the outer cannula may be distributed separately from the bidirectional cannula. However, other uses of the outer cannula are possible as well.

The portion between the intermediate opening and the proximal portion may have an essentially constant diameter or maximum width along the longitudinal axis of the cannula. Variations may be less than 10 percent or less than 5 percent of a maximum value.

The cannula, preferably the outer cannula, may comprise a kink in the intermediate portion. The kink may include an angle in the range of 80 degrees to 130 degrees, preferably 110 degrees. The intermediate opening of the outer cannula may be arranged at the kink. The kink may facilitate the insertion of the cannula from a jugular vein into the heart. Furthermore, the kink may facilitate the insertion of a further cannula from the inside of the (outer) cannula through the intermediate opening.

The intermediate portion may comprise a conical portion or a decreasing diameter portion which reduces its outer diameter at positions which are more distally than other positions of the decreasing diameter portion. The conical portion or the decreasing diameter portion may vary in outer diameter by at least 3 French or by at last 4 French or by at least 5 French, preferably by less than 10 French or by less than 8 French. The diameter may be measured in a cross section which is perpendicular to longitudinal axis of cannula. Instead of the diameter a maximum width may be used if the portions have a non-circular cross section. The conical portion or the decreasing diameter portion may allow to place a further intermediate opening of a cannula which is inserted into the (outer) cannula with an offset relative to the intermediate opening of the (outer) cannula. This may reduce erroneous placements of the inner cannula. Furthermore, space may be sufficient in order to allow easy outflow from an intermediate opening of the inner cannula through a distal tip of the outer cannula or vice versa for an outflow. Furthermore, a flap may be arranged within the intermediate opening of the inner cannula which may require some working space outside of the inner cannula.

The proximal portion of the outer cannula may have a larger diameter than the distal portion of the outer cannula, for instance in the range of 1 Fr to 4 Fr.

The conical portion or the decreasing diameter portion may be arranged distally adjacent to the kink and/or distally adjacent to the intermediate portion of the (outer) cannula. This may allow easy insertion of another cannula.

The conical portion or the decreasing diameter portion may be comparably short, for instance with a longitudinal length in the range of 2 cm (centimeter) to 5 cm or in the range of 3 cm to 4 cm.

The conical portion or the decreasing diameter portion may be arranged between the intermediate portion of the cannula and a distal portion of the cannula. The distal portion may comprise an essentially constant diameter/maximum width portion comprising a length of at least 5 cm, of at least 10 cm or of at least 15 cm, preferably less than 30 cm. The diameter or the maximum width may be constant along the longitudinal axis of the cannula. Variations may be less than 10 percent or less than 5 percent of a maximum value. Thus, the conical portion/decreased diameter portion may not be longer as necessary allowing introducing the outer cannula with less trauma and/or preventing damage to the vessels of the blood circuit.

The distal portion may comprise an outer diameter of at least 19 French (1 Fr (French) equals ⅓ mm (millimeter)), of at least 21 French, of at least 23 French, of at least 25 French, of at least 27 French or of at least 29 French, preferably of less than 33 French or less than 31 French. The outer diameter depends on the anatomy of the patient and/or on the medical application. A larger diameter may enable higher flow rates and/or better timing of the blood flow.

The distance between the proximal portion and the intermediate opening may be at least 20 cm or at least 25 cm, preferably less than 35 cm or less than 30 cm. Thus, the cannula may be used for a wide range of anatomies and/or medical applications. The reference point for the measurement may be the center of the intermediate opening of the (outer) cannula or an edge of intermediate opening which is closed to proximal portion of the (outer) cannula.

The (outer) cannula according to any one of the embodiments mentioned above, may be adapted to be used as an outer cannula in a method according to any one of the embodiments mentioned above. The same technical effects apply, e.g. new medical applications, less trauma, less mechanical stress to at least one blood vessel.

The features of outer cannula may also be valid for the outer cannula of the cannula set or assembly mentioned above and vice versa.

The basic principle of an endovascular catheter/cannula therapy may be a treatment of vessels and/or by using vessels for the advancement of a catheter, for instance plastic tubes or plastic tubes that are armed with metal. An incision may be made into the skin of a patient. The incision may have a length that is less than 5 cm (centimeter), less than 3 cm or less than 1 cm. Local anesthesia may be used thereby. An auxiliary cannula may be used to insert a guide wire and/or dilators to expand the incision and/or an opening within the vessel. The catheter or cannula may then be inserted using the guide wire and/or an introducing member.

No thoracotomy may be necessary if cannulas or catheters are used. A cannula may be a tube that can be inserted into the body, often for the delivery or removal of fluid or for the gathering of data. A catheter may be a thin tube made from medical grade materials serving a broad range of functions. Catheters may be medical devices that can be inserted into the body to treat diseases or to perform a surgical procedure. Both terms “cannula” and “catheter” are used interchangeably in the following if not stated otherwise. No special surgery may be necessary, i.e. it may not be necessary that a very high specialized physician or surgeon uses the proposed cannula and/or performs the proposed methods.

By modifying the material or adjusting the way cannulas or catheters are manufactured, it is possible to tailor them for cardiovascular, urological, gastrointestinal, neurovascular, and ophthalmic applications. A catheter or cannula may be left inside the body, either temporarily or permanently. A permanently catheter or cannula may be referred to as an “indwelling catheter or cannula” (for example, a peripherally inserted central catheter or cannula).

Catheters and cannulas may be inserted into a body cavity, duct or vessel. Functionally, they allow delivery and/or drainage of fluids, administration of fluids or gases, access by surgical instruments, and/or also perform a wide variety of other tasks depending on the type of catheter or cannula. The process of inserting a catheter is “catheterization”. The process of inserting a cannula is “cannulization”. In most uses, a catheter or cannula is a thin, flexible tube (“soft”) though catheters or cannulas are available in varying levels of stiffness depending on the application.

In this application document the definition for “distal” is far from a person that inserts the cannula or catheter. “Proximal” means near to the person that inserts the cannula or catheter. In the following the longitudinal axis of the lumen portion or the extension thereof beyond the lumen portion may be used as a reference axis. The terms “radial”, “axial” and/or “angularly” may be used with regard to this reference axis. This may be similar to the usage of cylinder coordinates that are used in a cylindrical coordinate system.

The proposed method and its embodiments may not be used for treatment of the human or animal body by surgery or therapy and may not be a diagnostic method practiced on the human or animal body. Alternatively, the proposed method and its embodiments may be used for treatment of the human or animal body by surgery or therapy and may be a diagnostic method practiced on the human or animal body.

The making and usage of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference signs refer to the same technical features if not stated otherwise. As far as “may” is used in this application it means the possibility of doing so as well as the actual technical implementation. The present concepts of the present disclosure will be described with respect to preferred embodiments below in a more specific context namely heart and/or lung surgery and/or support. The disclosed concepts may also be applied, however, to other situations and/or arrangements in heart surgery/support and/or lung surgery/support as well, especially to surgery and/or support of other organs.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

For a more complete understanding of the presently disclosed concepts and the advantages thereof, reference is now made to the following description in conjunction with the accompanying drawings. The drawings are not drawn to scale. In the drawings the following is shown in:

FIG. 1 a variant 1 for left heart support with puncturing of the atrial septum of the heart,

FIG. 2 a variant 2 for left heart support with puncturing of the atrial septum of the heart,

FIG. 3 a variant 3 for lung support with puncturing of the atrial septum of the heart,

FIG. 4 a variant 4 for transcaval heart and/or lung support,

FIG. 5 a variant 5 for heart and/or lung support with puncturing of the ventricle septum of the heart,

FIG. 6 a variant 6 for heart and/or lung support with puncturing of the ventricle septum of the heart,

FIG. 7 a renal support system using a single membrane pump and a bidirectional cannula with blood delivery at an intermediate portion,

FIG. 8 a renal support system using two membrane pumps in parallel and a bidirectional cannula with blood delivery at an intermediate portion,

FIG. 9 a pRVAD® support system using two membrane pumps in parallel (dual membrane pump) or a single membrane pump, and

FIG. 10 an ECCO₂R system with pulmonary drainage and delivery of blood into left atrium or into left ventricle.

FIG. 1 illustrates a variant 1 for a left heart H support with puncturing of an atrial septum AS of the heart H. Heart H comprises:
right atrium RA,
right ventricle RV,
left atrium LA,
left ventricle LV,
atrial septum AS between right atrium RA and left atrium LA, and
ventricle septum VS between right ventricle RV and left ventricle LV.

The following valves of heart H are shown in the following FIGS. 1 to 10:
tricuspid valve TV between right atrium RA and right ventricle RV, and
mitral valve MV between left atrium LA and left ventricle LV,
aortic valve 155, AV is between aorta AO and left ventricle LV, and
pulmonary valve 150, PV between right ventricle RV and pulmonary artery PA.

There are two left pulmonary veins lPV and two right pulmonary veins rPV that extend into left atrium LA of heart H. Blood that is enriched with oxygen comes from lung L into left atrium LA through pulmonary veins PV. This is an exception in that a vein transports blood that comprises more oxygen than blood in a comparable artery. The description of heart H will not be repeated below. However, it is clear that this description is valid for all FIGS. 1 to 6 that show heart H.

A cannula CA1 comprises:
a proximal portion PP1,
an intermediate portion IP1, and
a distal portion DP1.

Proximal portion PP1 may be connected to a pump or to a variable volume reservoir. An extracorporeal oxygenator may be inserted between cannula CA1 and the pump or the variable volume reservoir if lung L support is needed. However, left heart H support may be performed without oxygenation if no lung L support is needed, i.e. lung L of the patient is able to deliver enough oxygen for body 100.

Intermediate portion IP1 may comprise at least one intermediate opening IO1. Intermediate portion IP1 may be configured such that depending on the direction of the blood flow within proximal portion PP1 two different flows are generated within intermediate portion IP1 and within distal portion DP1. A simple solution for this flow selectivity is the usage of a two-way valve or of another valve configuration. Alternatively or additionally, an appropriate fluidically design of cannula CA1 may be used.

Distal portion DP1 may comprise at least one or exactly one distal opening DO1.

Optional expandable arrangements EA1a and EA1b may be used around intermediate opening IO1 and/or distal opening DO1. Expandable arrangements EA1a and/or EA1b may fulfill a fixation function and/or other functions as mentioned above.

If the blood flow within proximal part PP1 is directed proximally, blood is sucked or drained into (see arrow I) intermediate opening IO1 but not or only to a less degree through distal opening DO1. If the blood flow within proximal part PP1 is directed distally, blood is delivered out of distal opening DO1, see arrow O, but not or only to a less degree out of intermediate opening IO1.

The total length of cannula CA1 may be selected as mentioned above for variant 1. The distance between distal end of cannula CA1 and intermediate opening IO1 may be as mentioned above for variant 1.

Cannula CA1 may be inserted endovascularly and jugular through vena cava VC, preferably through superior vena cava SVC, right atrium RA, atrial septum AS, left atrium LA, left ventricle LV at least up to ascending aorta aAO, AO with blood drainage through intermediate opening IO1 from left atrium LA and with blood delivery out of distal opening DO1 into aorta AO.

FIG. 2 illustrates a variant 2 for left heart H support with puncturing of atrial septum AS of heart H. A cannula CA2 comprises:
a proximal portion PP2,
an intermediate portion IP2, and
a distal portion DP2.

Proximal portion PP2 may be connected to a pump or to a variable volume reservoir. An extracorporeal oxygenator may be inserted between cannula CA2 and the pump or the variable volume reservoir if lung L support is needed. However, left heart H support may be performed without oxygenation if no lung L support is needed, i.e. lung L of the patient is able to deliver enough oxygen for body 100.

Intermediate portion IP2 may comprise at least one intermediate opening IO2. Intermediate portion IP2 may be configured such that depending on the direction of the blood flow within proximal portion PP2 two different flows are generated within intermediate portion IP2 and within distal portion DP2. A simple solution for this flow selectivity is the usage of a two-way valve or of another valve configuration. Alternatively or additionally an appropriate fluidically design of cannula CA2 may be used.

Distal portion DP2 may comprise at least one or exactly one distal opening DO2.

Optional expandable arrangements may be used around intermediate opening IO2 and/or distal opening DO2. The expandable arrangements may fulfill a fixation function and/or other functions as mentioned above.

If the blood flow within proximal part PP2 is directed proximally, blood is sucked or drained into (see arrow I) intermediate opening IO2 but not or only to a less degree through distal opening DO2. If the blood flow within proximal part PP2 is directed distally, blood is delivered out of distal opening DO2, see arrow O, but not or only to a less degree out of intermediate opening IO2.

The total length of cannula CA2 may be selected as mentioned above for variant 2. The distance between distal end of cannula CA2 and intermediate opening IO2 may be as mentioned above for variant 2.

Cannula CA2 may be inserted endovascularly and jugular through vena cava VC, preferably through superior vena cava VC, right atrium RA, atrial septum AS, left atrium LA, left ventricle LV at least up to the ascending aorta aAO, AO with blood drainage through intermediate opening IO2 from left ventricle LV and with blood delivery out of distal opening DO2 into the aorta AO.

FIG. 3 illustrates a variant 3 for lung L support with puncturing of atrial septum AS of heart H. A cannula CA3 comprises:
a proximal portion PP3,
an intermediate portion IP3, and
a distal portion DP3.

Proximal portion PP3 of cannula CA3 may be connected to a pump or to a variable volume reservoir MP3. An extracorporeal oxygenator device OXY3 may be inserted between cannula CA3 and the pump or variable volume reservoir MP3 if lung L support is needed. However, left heart H support may be performed without oxygenation if no lung L support is needed, i.e. the lung L of the patient is able to deliver enough oxygen for body 100.

Oxygenator OXY3 may be a commercially available oxygenator. A blood filter unit may be used in addition to oxygenator OXY3. Medicaments/drugs or other treatment substances may be given or administered by an optional drug delivery unit that may be included into the fluidic circuitry that is shown in FIG. 3.

An assembly A3 comprises cannula CA3 and variable volume reservoir MP3. Variable volume reservoir MP3 may be arranged as near as possible to body 100 of a patient. Variable volume reservoir MP3 may comprise:
a rigid housing or casing,
a flexible membrane M within the housing,
one port for blood transport or two ports for blood inflow and blood outflow, and
a least one port Po3, that is connected with a fluid reservoir, for instance with a gas reservoir, especially with a helium or with an air reservoir.

If reservoir MP3 has only one port for blood transport, oxygenator OXY3 may be coupled fluidically between this port and cannula CA3. Alternatively, reservoir MP3 may have two ports for blood transport as shown in FIG. 3. Proximal portion PP3 of cannula CA3 may be connected to a connector that realizes a bifurcation, for instance to a Y-connector or to a T-connector. A first branch of the fluidic circuitry may be between proximal portion PP3 of cannula CA3 and a first blood port of reservoir MP3, see arrow Dir3a. A second branch of the fluidic circuitry may be between the second blood port of reservoir MP3 and proximal portion PP3 of cannula CA3, see arrow Dir3b. Both ports may comprise valves that make sure that the blood does only flow in the direction indicated by arrows Dir3a and Dir3b although membrane M moves back and forth only. As shown in FIG. 3, oxygenator OXY3 is included within the backflow branch relative to reservoir MP3. However, oxygenator OXY3 may also be included in the other branch or two oxygenators may be used in both branches.

Port Po3 may be connected to a piston arrangement or to another arrangement that is able to pump gas or another fluid, for instance a liquid, in and out of the housing of reservoir MP3. The piston arrangement or the other arrangement may be controlled depending on the heartbeat of the patient, e.g. based on electrocardiography (ECG) signals or data or other sensor signals or data. Preferably the diastole of left ventricle LV may be used to drain blood into aorta AO. However, other timing schemes are possible as well.

Intermediate portion IP3 of cannula CA3 may comprise at least one intermediate opening IO3. Intermediate portion IP3 may be configured such that two different flows are generated in intermediate portion IP3 and in distal portion DP3 depending on the direction of the blood flow in proximal portion PP3. A simple solution for this flow selectivity is the usage of a two-way valve or of another valve configuration. Alternatively or additionally an appropriate fluidically design of cannula CA3 may be used.

Distal portion DP3 may comprise at least one or exactly one distal opening DO3, for instance in combination with an expandable arrangement, e.g. a cage arrangement.

Optional expandable arrangements may be used around intermediate opening IO3 and/or distal opening DO3. The expandable arrangements may fulfill a fixation function and/or other functions as mentioned above.

If the blood flow in proximal part PP3 is directed proximally, blood is sucked or drained into (see arrow I) intermediate opening IO3 but not or only to a less degree through distal opening DO3. If the blood flow in proximal part PP3 is directed distally, blood is delivered out of distal opening DO3, see arrow O, but not or only to a less degree out of intermediate opening IO3.

The total length of cannula CA3 may be as mentioned above for variant 3. The distance between distal end of cannula CA3 and intermediate opening IO3 may be as mentioned above for variant 3.

Cannula CA3 may be inserted endovascularly and jugular through vena cava VC, right atrium RA, atrial septum AS, left atrium LA, left ventricle LV at least up to the ascending aorta aAO, AO. Blood may be drained through intermediate opening IO3 from right atrium RA if the membrane M increases the volume of reservoir MP3. This blood flows in the first branch along direction Dir3a into reservoir MP3. Blood is delivered out of reservoir MP3 if the membrane M decreases the volume of reservoir MP3. This blood flows through the second branch, i.e. along direction Dir3b into cannula CA3 and is delivered out of distal opening DO3 into aorta AO. Within the next pumping cycle this is repeated. Alternatively, reservoir MP3 may have only one port for blood transfer and oxygenator OXY3 may be an oxygenator for bidirectional flow, i.e. no bifurcation element is needed.

Alternatively, the inlet opening may be arranged within vena cava VC, i.e. the distance between the distal end of cannula CA3 and intermediate opening IO3 has to be increased, for instance by a value within the range of 2.5 cm to 7.5 cm, preferably by 5 cm. The length of cannula CA3 may be the same independent of the location of intermediate opening IO3 in vena cava VC or in right atrium RA.

FIG. 4 illustrates a variant 4 for transcaval heart H and/or lung L support. A cannula CA4 comprises:
a proximal portion PP4,
an intermediate portion IP4, and
a distal portion DP4.

Proximal portion PP4 of cannula CA4 may be connected to a pump or to a variable volume reservoir MP4. An extracorporeal oxygenator OXY4 may be inserted between cannula CA4 and the pump or variable volume reservoir MP4 for lung L support.

Oxygenator OXY4 may be a commercially available oxygenator. A blood filter unit may be used in addition to the oxygenator OXY4. Medicaments/drugs or other treatment substances may be given or administered by an optional drug delivery unit that may be included into the fluidic circuitry that is shown in FIG. 4.

An assembly A4 may comprise cannula CA4 and variable volume reservoir MP4 or a pump. Variable volume reservoir MP4 may be arranged as near as possible to body 100 of a patient. Variable volume reservoir MP4 may comprise:
a rigid housing or casing,
a flexible membrane M within the housing,
one port for blood transport or two ports for blood inflow and blood outflow, and
at least one port Po4, that is connected with a fluid reservoir, for instance with a gas reservoir, especially helium or air reservoir.

If reservoir MP4 has only one port for blood transport, oxygenator OXY4 may be coupled fluidically between this port and cannula CA4. Alternatively, reservoir MP4 may have two ports for blood transport as shown in FIG. 4. Proximal portion PP4 of cannula CA4 may be connected to a connector that realizes a bifurcation, for instance to a Y-connector or to a T-connector. A first branch of the fluidic circuitry may be between proximal portion PP4 of cannula CA4 and a first blood port of reservoir MP4, see arrow Dir4a. A second branch of the fluidic circuitry may be between the second blood port of reservoir MP4 and the proximal portion PP4 of cannula CA4, see arrow Dir4b. Both ports may comprise valves that make sure that the blood does only flow in the direction indicated by arrows Dir4a and Dir4b although membrane M moves back and forth only. As shown in FIG. 4, oxygenator OXY may be included within the backflow branch relative to reservoir MP4. However, oxygenator OXY4 may also be included in the other branch or two oxygenators may be used in both branches.

Port Po4 may be connected to a piston arrangement or to another arrangement that is able to pump gas or another fluid, for instance a liquid, in and out of the housing of reservoir MP4. The piston arrangement or the other arrangement may be controlled depending on the heartbeat of the patient, e.g. based on electrocardiography (ECG) signals or data or other sensor signals or data. Preferably the diastole of left ventricle LV may be used to drain blood into Aorta AO. However, other timing schemes are possible as well.

Intermediate portion IP4 of cannula CA3 may comprise at least one intermediate opening IO4. Intermediate portion IP4 may be configured such that two different flows are generated in intermediate portion IP4 and in distal portion DP4 depending on the direction of the blood flow in proximal portion PP4. A simple solution for this flow selectivity is the usage of a two-way valve or of another valve configuration. Alternatively or additionally, an appropriate fluidically design of cannula CA4 may be used.

The distal portion DP4 may comprise at least one or exactly one distal opening DO4, for instance in combination with an expandable arrangement, e.g. a cage arrangement.

Optional expandable arrangements may be used around intermediate opening IO4 and/or distal opening DO4. The expandable arrangements may fulfill a fixation function and/or other functions as mentioned above.

If the blood flow in proximal part PP4 is directed proximally, blood is sucked or drained into (see arrow I) intermediate opening IO4 but not or only to a less degree through distal opening DO4. If the blood flow in the proximal part PP4 is directed distally, blood is delivered out of distal opening DO4, see arrow O, but not or only to a less degree out of intermediate opening IO4.

The total length of cannula CA4 may be as mentioned above for variant 4. The distance between distal end of cannula CA4 and intermediate opening IO4 may be as mentioned above for variant 4.

Cannula CA4 may be inserted endovascularly and jugular through vena cava VC, preferably through superior vena cava SVC, to right atrium RA and punctured transcaval directly from right atrium RA into aorta AO and then inserted up to the ascending aorta aAO, AO. Blood may be drained through intermediate opening IO4 from right atrium RA if membrane M increases the volume of reservoir MP4. This blood flows in the first branch along direction Dir4a into reservoir MP4. Blood is delivered out of reservoir MP4 if membrane M decreases the volume of reservoir MP4. This blood flows through the second branch, i.e. along direction Dir4b into cannula CA4 and is delivered out of distal opening DO4 into aorta AO. Within the next pumping cycle this is repeated. Alternatively, reservoir MP4 may have only one port for blood transfer and oxygenator OXY4 may be an oxygenator for bidirectional flow, i.e. no bifurcation element is needed.

Alternatively, the inlet opening may be arranged within vena cava VC, i.e. the distance between the distal end of cannula CA4 and intermediate opening IO4 has to be increased, for instance by a value within the range of 2.5 cm to 7.5 cm, preferably by 5 cm. The length of cannula CA4 may be the same independent of the location of intermediate opening IO4 in vena cava VC or in right atrium RA.

Variant 4b: A cannula CA4a may be similar to cannula CA4. However, the difference is that cannula CA4a is inserted endovascular, preferably jugular, up to vena cava VC and then punctured directly transcaval from vena cava VC directly to aorta AO, for instance to ascending aorta aAO. An intermediate opening IO4a may be located within vena cava VC and may be used for blood drainage or blood removal from vena cava VC. Blood delivery remains into aorta AO, preferably into ascending aorta AO. At least one oxygenator may be used, for instance coupled into a one directional flow or a bi-directional flow of the fluid circuitry. The length of cannula CA4a may be the same as the length of cannula CA4. The distance between the distal end and the intermediate opening IO4a may be increased for cannula CA4a if compared to the same distance at cannula CA4 in the range of 2.5 cm to 7.5 cm, preferably by 5 cm.

Variant 10: A cannula CA10 may be coupled to variable volume reservoir MP4 in the same way as cannula CA4. Alternatively, the oxygenator may be coupled to a reservoir MP4 having only one port for blood transport. In another embodiment for cannula CA10 no oxygenator may be used. However in both cases (i.e. with or without oxygenator), cannula CA10 may be inserted endovascular, preferably jugular, up to vena cava VC, then to right atrium RA and then punctured directly, i.e. transcaval, from right atrium RA directly into pulmonary artery PA, especially into main pulmonary artery PA. The length of cannula CA10 may be the length that is mentioned above in item a10). The distance between the distal tip and the intermediate opening IO10 of cannula 10 may be the distance that is mentioned above in item a10). Blood drainage may be made from vena cava VC or from right atrium RA.

Variant 10b: A cannula CA10a may be similar to cannula CA10. However, the difference is that cannula CA10a is inserted endovascular, preferably jugular, up to vena cava VC and then punctured directly from vena cava VC transcaval to pulmonary artery PA. An intermediate opening IO10a may be located within vena cava VC and may be used for blood drainage from vena cava VC. Blood delivery may remain into aorta AO, preferably into ascending aorta AO. At least one oxygenator may be used, for instance coupled into a one directional flow or a bi-directional flow of the fluid circuitry. The length of cannula CA10a may be the same as the length of cannula CAb0. The distance between the distal end and the intermediate opening may be increased for cannula CA10a if compared to the same distance at cannula CA10 in the range of 2.5 cm to 7.5 cm, preferably by 5 cm.

FIG. 5 illustrates a variant 5 for heart H and/or lung L support with puncturing of ventricle septum VS of heart H. A cannula CA5 comprises:
a proximal portion PP5,
an intermediate portion IP5, and
a distal portion DP5.

Proximal portion PP5 may be connected to a pump or to a variable volume reservoir. An extracorporeal oxygenator may be inserted between cannula CA5 and the pump or the variable volume reservoir if lung support is needed. However, left heart support may be performed without oxygenation if no lung support is needed, i.e. the lung of the patient is able to deliver enough oxygen for body 100.

Intermediate portion IP5 may comprise at least one intermediate opening IO5. Intermediate portion IP5 may be configured such that depending on the direction of the blood flow in the proximal portion PP5 two different flows are generated in the intermediate portion IP5 and in the distal portion DP5. A simple solution for this flow selectivity is the usage of a two-way valve or of another valve configuration. Alternatively or additionally, an appropriate fluidically design of cannula CA5 may be used.

Distal portion DP5 may comprise at least one or exactly one distal opening DO5.

Optional expandable arrangements may be used around intermediate opening IO5 and/or distal opening DO5. The expandable arrangements may fulfill a fixation function and/or other functions as mentioned above.

If the blood flow in proximal part PP5 is directed proximally, blood is sucked or drained into (see arrow I) intermediate opening IO5 but not or only to a less degree through distal opening DO5. If the blood flow in proximal part PP5 is directed distally, blood is delivered out of distal opening DO5, see arrow O, but not or only to a less degree out of intermediate opening IO5.

The total length of cannula CA5 may be as mentioned above for variant 5. The distance between distal end of cannula CA5 and intermediate opening IO5 may be as mentioned above for variant 5.

Cannula CA5 may be inserted endovascularly and jugular through vena cava VC, right atrium RA, right ventricle RV, ventricle septum VS, left ventricle LV at least up to ascending aorta aAO, AO with blood drainage through intermediate opening IO5 from left ventricle LV and with blood delivery out of distal opening DO5 into the aorta AO.

FIG. 6 illustrates a variant 6 for heart H and/or lung L support with puncturing of the ventricle septum VS of heart H. A cannula CA6 comprises:
a proximal portion PP6,
an intermediate portion IP6, and
a distal portion DP6.

Proximal portion PP6 of cannula CA6 may be connected to a pump or to a variable volume reservoir MP6. An extracorporeal oxygenator OXY6 may be inserted between cannula CA6 and the pump or the variable volume reservoir if lung L support is needed. However, left heart H support may be performed without oxygenation if no lung L support is needed, i.e. lung L of the patient is able to deliver enough oxygen for body 100.

Oxygenator OXY6 may be a commercially available oxygenator. A blood filter unit may be used in addition to the oxygenator OXY6. Medicaments/drugs or other treatment substances may be given or administered by an optional drug delivery unit that may be included into the fluidic circuitry that is shown in FIG. 6.

An assembly A6 may comprise cannula CA6 and variable volume reservoir MP6. Variable volume reservoir MP6 may be arranged as near as possible to body 100 of a patient. Variable volume reservoir MP6 may comprise:
a rigid housing or casing,
a flexible membrane M within the housing,
one port for blood transport or two ports for blood inflow and blood outflow, and
at least one port Po6, that is connected with a fluid reservoir, for instance with a gas reservoir, especially helium or air reservoir.

If reservoir MP6 has only one port for blood transport, oxygenator OXY6 may be coupled fluidically between this port and cannula CA6. Alternatively, reservoir MP6 may have two ports for blood transport as shown in FIG. 6. Proximal portion PP6 of cannula CA6 may be connected to a connector that realizes a bifurcation, for instance to a Y-connector or to a T-connector. A first branch of the fluidic circuitry may be between proximal portion PP6 of cannula CA6 and a first blood port of reservoir MP6, see arrow Dir6a. A second branch of the fluidic circuitry may be between the second blood port of reservoir MP6 and the proximal portion PP6 of cannula CA6, see arrow Dir6b. Both ports may comprise valves that make sure that the blood does only flow in the direction indicated by arrows Dir6a and Dir6b although membrane M moves back and forth only. As shown in FIG. 6, oxygenator OXY6 is included within the backflow branch relative to reservoir MP6. However, oxygenator OXY6 may also be included in the other branch or two oxygenators may be used in both branches.

Port Po6 may be connected to a piston arrangement or to another arrangement that is able to pump gas or another fluid, for instance a liquid, in and out of the housing of reservoir MP6. The piston arrangement or the other arrangement may be controlled depending on the heartbeat of the patient, e.g. based on electrocardiography (ECG) signals or data or other sensor signals or data. Preferably the diastole of left ventricle LV may be used to drain blood into aorta AO. However, other timing schemes are possible as well.

Intermediate portion IP6 of cannula CA6 may comprise at least one intermediate opening IO6. The intermediate portion IP6 may be configured such that depending on the direction of the blood flow in the proximal portion PP6 two different flows are generated in intermediate portion IP6 and in distal portion DP6. A simple solution for this flow selectivity is the usage of a two-way valve or of another valve configuration. Alternatively or additionally, an appropriate fluidically design of cannula CA6 may be used.

Distal portion DP6 may comprise at least one or exactly one distal opening DO6, for instance in combination with an expandable arrangement, e.g. a cage arrangement.

Optional expandable arrangements may be used around intermediate opening IO6 and/or distal opening DO6. The expandable arrangements may fulfill a fixation function and/or other functions as mentioned above.

If the blood flow in the proximal part PP6 is directed proximally, blood is sucked or drained into (see arrow I) intermediate opening IO6 but not or only to a less degree through distal opening DO6. If the blood flow in the proximal part PP6 is directed distally, blood is delivered out of distal opening DO6, see arrow O, but not or only to a less degree out of intermediate opening IO6.

The total length of cannula CA6 may be as mentioned above for variant 6. The distance between distal end of cannula CA6 and intermediate opening IO6 may be as mentioned above for variant 6.

Cannula CA6 may be inserted endovascularly and jugular through vena cava VC, preferably through superior vena cava SVC, right atrium RA, right ventricle RV, ventricle septum VS, left ventricle LV at least up to the ascending aorta aAO, AO. Blood may be drained into intermediate opening IO6 from right atrium RA if membrane M increases the volume of reservoir MP6. This blood flows in the first branch along direction Dir6a into reservoir MP6. Blood is delivered out of reservoir MP6 if membrane M decreases the volume of reservoir MP6. This blood flows through the second branch, i.e. along direction Dir6b into cannula CA6 and is delivered out of distal opening DO6 into the aorta AO. Within the next pumping cycle this is repeated.

Alternatively, the inlet opening may be arranged within vena cava VC, i.e. the distance between the distal end of cannula CA6 and intermediate opening IO6 has to be increased appropriately. The length of cannula CA6 may be the same independent of the location of intermediate opening IO6 in vena cava VC or in right atrium RA.

Furthermore, alternatively, the inlet opening may be arranged within right ventricle RV, i.e. the distance between the distal end of cannula CA6 and intermediate opening IO6 has to be decreased appropriately. The length of cannula CA6 may be the same independent of the location of intermediate opening IO6 in right ventricle RV or in right atrium RA.

Further variants with delivery of blood into pulmonary artery PA, for instance right heart H support and/or lung L support:

Variant 7: A cannula CA7 that has an intermediate portion that is similar to intermediate portion IP6 may be inserted endovascularly and jugular through vena cava VC, preferably superior vena cava SVC, right atrium RA, right ventricle RV at least up to pulmonary artery PA. Blood may be drained through intermediate opening IO7 from right atrium RA if the membrane M increases the volume of reservoir MP6. This blood flows in the first branch along direction Dir6a into reservoir MP6. Blood may be delivered out of reservoir MP6 if the membrane M decreases the volume of reservoir MP6. This blood flows through the second branch, i.e. along direction Dir6b into cannula CA7 and is delivered out through distal opening DO7 into pulmonary artery PA. Within the next pumping cycle this is repeated. Alternatively, reservoir MP6 may have only one port for blood transfer and oxygenator OXY6 may be an oxygenator for bidirectional flow, i.e. no bifurcation element is needed.

Alternatively, no oxygenator OXY6 may be used, for instance in no lung L support is needed for variant 7.

Variant 8: Same as variant 7 but drainage from right ventricle RV. The total length of a cannula CA8 may be the same as the total length of cannula CA7 but the distance between the distal tip and the intermediate opening may be reduced appropriately as mentioned in the first part of the description and in the claims.

Variant 9: Same as variant 7 but drainage from vena cava VC. The total length of a cannula CA9 may be the same as the total length of cannula CA7 but the distance between the distal tip and the intermediate opening may be increased appropriately as mentioned in the first part of the description and in the claims.

Variant 11: A cannula CA11 that has a modified intermediate part may be used. The cannula CA11 of variant 11 may have a modified intermediate portion IP11. The intermediate portion IP1I of cannula CA11 may comprise at least one intermediate opening IO11. The intermediate portion IP11 may be configured such that depending on the direction of the blood flow in a proximal portion PP11 of cannula CA11 two different flows are generated in the intermediate portion IP11 and in a distal portion DP11 of cannula CA11. A simple solution for this flow selectivity is the usage of a two-way valve or of another valve configuration. Alternatively or additionally, an appropriate fluidically design of cannula CA11 may be used.

If the blood flow in the proximal part PP11 is directed distally, blood is delivered out of intermediate opening IO11, but not or only to a less degree through distal opening DI11. If the blood flow in the proximal part PP11 is directed proximally, blood is sucked or drained into distal opening DI3 but not or only to a less degree through intermediate opening IO11.

It may be possible to inverse the operating directions of cannula CA11 compared to the operating directions of cannula CA1 to CA10, for instance by changing the assembly direction of a valve or of several valves that are used within cannulas CA1 to CA10 mentioned above.

Cannula CA11 may be inserted endovascularly, for instance through a subclavian vein into the aorta and further into left ventricle LV. Blood may be drained out of left ventricle LV and delivered into aorta AO, preferably into ascending aorta aAO. Cannula CA11 may have a distal expandable arrangement and/or an expandable arrangement at the intermediate opening.

An oxygenator may be used together with the arrangement of cannula CA11. Alternatively no oxygenator may be used. Other medical applications of cannula CA11 with or without the usage of an oxygenator are possible as well.

FIG. 7 illustrates a renal support system 700 using a single membrane pump MP7 and a bidirectional cannula CA107 with blood delivery at an intermediate portion IP107. A distal portion DP107 of the bidirectional cannula CA107 may be inserted endovascularly, preferably jugularly, through superior vena cava SVC, right atrium RA and inferior vena cava IVC at least to or to a location which has to the junction of the renal veins rV1, rV2 into the inferior vena cava IVC a distance equal to 10 cm (centimeter) or less than 10 cm, equal to 5 cm or less than 5 cm or equal to 2.5 cm or less than 2.5 cm. Blood may be drained into at least one distal opening DO107, see arrows A107a, A107c and A107d. Thereafter the blood is sucked to the membrane pump(s) MP7 in an aspiration phase. Blood is expulsed into cannula CA107 during an expulsion phase. The expulsed blood may be delivered out of the at least one intermediate opening IO107 of cannula CA107 into the right atrium RA, see arrow A107b. Thus, blood may be pulled from the kidney(s). Only one membrane pump MP7 may be used which may be coupled to an intra-aortic balloon pump console IABP7. IABP console IABP7 may be controlled by electric impulses of heart H.

Membrane pump MP7 may be a one port membrane pump which may comprise or have only one port for liquid transport into the membrane pump MP7 and our of membrane pump MP7.

Bidirectional cannula CA107 may be visible in an X-ray device or within another medical image generating device. Using the image generating device, the intermediated opening may be aligned such that it is directed to the center of the right atrium RA thus resulting in flow out of the intermediate opening IO107 which is directed directly to the tricuspid valve TV. A complete antegrad outflow is generated therewith, i.e. a blood flow which is directed in the natural flow directions of the blood. Opposite pulsation is avoided which results in less unnatural turbulences. The same may be valid for other bidirectional cannulas CA108, CA109a and CA110a mentioned below.

There may be the following dimensions of cannula CA107:

| Body height of | 110 cm | 140 | cm | 160 | cm | 180 | cm | 200 cm |
|---|---|---|---|---|---|---|---|---|
| patient | | | | | | | | |
| Distance | 5 cm to 10 cm | 10 cm to 15 cm | | 15 cm to 20 cm | | 20 cm to 25 cm | | 25 cm to 30 cm |
| between IO107 | | | | | | | | |
| and DP107 | | | | | | | | |
| Outer diameter, | 19 Fr | 21 | Fr | 23 | Fr | 25 | Fr | 27 Fr or 29 Fr |
| for instance at | (French) | | | | | | | |
| PP107 and/or at | | | | | | | | |
| DP107 | | | | | | | | |

FIG. 8 illustrates a renal support system 800 using two membrane pumps MP8a, MP8b in parallel and a bidirectional cannula CA108 with blood delivery at an intermediate portion IP108. All other parts correspond to parts mentioned in FIG. 7, e.g.:
cannula CA108 to cannula CA107,
proximal portion PP108 to PP107,
intermediate portion IP108 to IP107,
intermediate opening IO108 to IO107,
distal portion DP108 of cannula CA108 to distal portion DP107 of cannula CA107,
distal opening DO108 to DO107, and
arrows A108a to A108d to arrows A107a to A107d.

There may be the following dimensions of cannula CA108:

| Body height of | 110 cm | 140 | cm | 160 | cm | 180 | cm | 200 cm |
|---|---|---|---|---|---|---|---|---|
| patient | | | | | | | | |
| Distance | 5 cm to 10 cm | 10 cm to 15 cm | | 15 cm to 20 cm | | 20 cm to 25 cm | | 25 cm to 30 cm |
| between IO108 | | | | | | | | |
| and DP108 | | | | | | | | |
| Outer diameter, | 19 Fr | 21 | Fr | 23 | Fr | 25 | Fr | 27 Fr or 29 Fr |
| for instance at | (French) | | | | | | | |
| PP108 and/or at | | | | | | | | |
| DP108 | | | | | | | | |

Support system 800 may use two membrane pumps MP8a or MP8b in parallel (dual membrane pump) or a single membrane pump. Membrane pumps MP8a and MP8b may be coupled to the same port of an IABP console IABP8. IABP console IABP8 may be controlled by electrical signals of heart H. Both membrane pumps MP8a or MP8b may be connected to a proximal portion PP108 of bidirectional cannula CA108 via a branch B8 and a connection C8. Branch B8 may be a Y-connector, a T-connector or another three-port element.

The function of arrangement 800 is similar to the function of arrangement 700, e.g. renal support is possible.

FIG. 9 illustrates a pRVAD® (percutaneous right ventricle assist device) support system 900 using two membrane pumps MP9a or MP9b in parallel (dual membrane pump) or a single membrane pump MP9c. Membrane pumps MP9a and MP9b may be coupled to the same port of an IABP console IABP9. IABP console IABP9 may be controlled by electrical signals of heart H. Both membrane pumps MP9a or MP9b may be connected to a proximal portion PP109a of a bidirectional cannula CA109a via a branch B9 and a connection C9. Branch B9 may be a Y-connector, a T-connector or another three-port element.

Support system 900 may comprise a cannula system CS. Cannula system CS may comprise or may consist of the bidirectional cannula CA109a and an outer cannula CA109b. Bidirectional cannula CA109a may comprise:
a proximal portion PP109a,
an intermediate portion IP109a,
an intermediate opening IO109a,
a distal portion DP109a, and
a distal opening DO109a.

Outer cannula CA109b may comprise:
a proximal portion PP109b,
a distal portion DP109b that comprises at least one distal opening DO109b,
a lumen portion LP that extends from the proximal portion PP109b to the at least one distal opening DO109b, and
at least one intermediate portion IP109b that is arranged between the proximal portion PP109b and the distal portion DP109b.

The intermediate portion IP109b of the outer cannula CA109b may comprise at least one lateral intermediate opening IO109b which may be configured to allow passage of the distal portion DP109a of the bidirectional cannula CA109a.

Before inserting the bidirectional cannula CA109a into the body of the patient, outer cannula CA109b may be inserted through the vessel of the blood circuit. Distal portion DP109b of outer cannula CA109b may be inserted endovascularly, preferably jugularly, through superior vena cava SVC, right atrium RA and right ventricle RV at least to the pulmonary artery PA,

Thereafter, the bidirectional cannula CA109a may be inserted into a first part FP-LP of the lumen portion of the outer cannula CA109b until the distal portion DP109a of the bidirectional cannula CA109a extends through the intermediate opening IO109b of the outer cannula CA109b and the intermediate opening IO109a of the bidirectional cannula CA109a is arranged within and/or aligned with the intermediate portion IP109b of the bidirectional cannula CA109a thereby being in fluidic connection with the distal portion DP109b of the outer cannula CA109b via a second part SP-LP of the lumen portion of the outer cannula CA109b. The distal portion DP109a of the bidirectional cannula CA109a is inserted into right atrium RA optionally further into inferior vena cava IVC.

Thereby, a first part FP-LP of the lumen portion of the outer cannula CA109b is formed between an inner surface of outer cannula CA109b and an outer surface of bidirectional cannula CA109a is formed. Furthermore, a second part SP-LP of the lumen portion of the outer cannula CA109b is formed between the intermediate opening IO109a of the bidirectional cannula CA109a and the distal portion DP109b of the outer cannula CA109b. The first part FP-LP of the lumen portion of the outer cannula CA109b may form a “dead” lumen portion.

Blood is drained into the at least one distal opening DO109a of the bidirectional cannula CA109a, see arrow 109a. Blood is transported in an aspiration phase into membrane pumps MP9a, MP9b; MP9c. In an expulsion phase blood is transported in the opposite direction. Due to the valves within bidirectional cannula CA109a or due to a specific fluidic design, blood is delivered out of the at least one intermediate opening IO109a of bidirectional cannula CA109a in the expulsion phase, see arrow A109b. Blood is delivered further through the at least one distal opening DO109b of the outer cannula CA109b, see arrow A109c. Usage of an oxygenator is optional in pVRAD® system 900.

Although outer cannula CA109b is illustrated with different diameters, especially in the intermediate portion IP109b it is of course also possible to have a constant diameter along the longitudinal axis of outer cannula CA109b.

Furthermore, outer cannula CA109b may have a kink K in the intermediate portion. In a state without outer forces (base state) the kink K may include an angle in the range of 80 degrees to 130 degrees, preferably 110 degrees. The kink K may facilitate the insertion of the distal portion DP109a of bidirectional cannula CA109a through intermediate opening IO109b of outer cannula CA109b. However, it is also possible to use an outer cannula without a kink K. Intermediate opening IO109b of outer cannula CA109b may be arranged at kink K, preferably in a distance from the kink which is less than 3 cm or less than 2 cm or less than 1 cm.

The bidirectional cannula CA109a may be essentially straight or straight if no external forces are applied. This may be true also for bidirectional cannulas CA107 and CA108 which are mentioned above.

With regard to valves V9a, V9b and V9c see description at the end of the description of FIG. 10.

There may be the following dimensions of cannula CA109a:

| Body height of | 110 | cm | 140 | cm | 160 | cm | 180 | cm | 200 cm |
|---|---|---|---|---|---|---|---|---|---|
| patient | | | | | | | | | |
| Distance | 1 cm to 5 cm or | | 1 cm to 5 cm or | | 1 cm to 5 cm or | | 1 cm to 5 cm or | | 1 cm to 5 cm or |
| between IO109a | 1 cm to 3 cm | | 1 cm to 3 cm | | 1 cm to 3 cm | | 1 cm to 3 cm | | 1 cm to 3 cm |
| and DP109a | | | | | | | | | |
| Outer diameter, | 19 | Fr | 21 | Fr | 23 | Fr | 25 | Fr | 27 Fr or 29 Fr |
| for instance at | | | | | | | | | |
| PP109a and/or | | | | | | | | | |
| at DP109b | | | | | | | | | |

The distal portion DP109b of the outer cannula CA109b may have the same outer diameter as the distal portion DP109a of the bidirectional cannula CA109a. Alternatively, the distal portion DP109b of the outer cannula CA109b may have a greater outer diameter than the distal portion DP109a of the bidirectional cannula CA1009a, for instance greater by at least 2 French but not greater than 8 French compared to the outer diameter of the distal portion DP109a of the bidirectional cannula CA109a. Alternatively, greater by at least 3 French but not greater than 8 French.

There may be the following dimensions of outer cannula CA109b:

| | | | | | |
|---|---|---|---|---|---|
| Body height of | 110 cm | 140 cm | 160 cm | 180 cm | 200 cm |
| patient | | | | | |
| Length of | 40 cm | 50 cm | 60 cm | 70 cm | 80 cm |
| cannula | (plus max. 5 cm | (plus max. 5 cm | (plus max. 5 cm | (plus max. 5 cm | (plus max. 5 cm |
| CA109b | and/or minus | and/or minus | and/or minus | and/or minus | and/or minus |
| | max. 5 cm) | max. 5 cm) | max. 5 cm) | max. 5 cm) | max. 5 cm) |
| Distance | 8 cm | 13 cm | 18 cm | 23 cm | 28 cm |
| IO109b to | 6 cm to 10 cm | 11 cm to 15 cm | 16 cm to 20 cm | 21 cm to 25 cm | 24 cm to 30 cm |
| DP109b | | | | | |

A conical portion ConP of outer cannula CA1009b may have a length of less than 10 cm for all cases, e.g. all body height. The conical portion ConP may have a reduction in diameter of at least 4 French of at least 5 French but preferably of not more than 8 French or of not more than 10 French. The conical portion ConP may be located distally adjacent to kink K and/or distally adjacent to intermediate portion JP109b.

The function of arrangement 900 is as follows. In an aspiration phase of the membrane pumps MP9a, MP9b or of the membrane pump MP9c blood is sucked from right atrium RA and/or inferior vena cava IVC into or drained into distal opening DO109a of bidirectional cannula CA109a into the variable volume reservoir of the membrane pump MP9a, MP9b or MP9c, see arrow A119a. In an expulsion phase of the membrane pumps MP9a, MP9b or of the membrane pump MP9c blood is expulsed or delivered out of intermediated opening IO109a of bidirectional cannula CA109a through a second part SP-LP of the outer cannula CA109b and out of distal opening DO109b of outer cannula CA109b into pulmonary artery PA. Thereafter, the cycle of aspiration phase and expulsion phase is repeated, for instance synchronous to the beats of heart H.

FIG. 10 illustrates an ECCO₂R (extracorporeal carbon dioxide removal) system 1000 with pulmonary drainage and delivery of blood into left atrium LA or into left ventricle LV. A cannula system CS may be used that comprises two cannulas CA110a and CA110b. A bidirectional cannula CA110a may comprise:
a proximal portion PP110a,
an intermediate portion IP110a,
an intermediate opening IO110a,
a distal portion DP110a, and
a distal opening DO110a.

An outer cannula CA110b may comprise:
a proximal portion PP110b,
a distal portion DP11Gb1, DP110b2 that comprises at least one distal opening DO110b1 or DP110b2,
a lumen portion LP that extends from the proximal portion PP110b to the at least one distal opening DO110b1 or DPb102, and
at least one intermediate portion IP110b that is arranged between the proximal portion PP110b and the distal portion DP110b1 or DP110b2.

The intermediate portion IP110b of the outer cannula CA11 Ob may comprise at least one lateral intermediate opening IO110b or exactly one lateral intermediate opening IO110b which is configured to allow passage of the distal portion DP110a of the bidirectional cannula CA110a.

Again, the outer cannula CA110b may be inserted before bidirectional cannula CA110a is inserted into the body of the patient. In a first alternative (variant 1), the distal portion DP110b1 of the outer cannula CA110b is inserted endovascularly, preferably jugularly, through superior vena cava SVC, right atrium RA and atrial septum AS up to the left atrium LA of the heart H.

In a second alternative (variant 2), the distal portion DP110b2 of the outer cannula CA110b may be inserted endovascularly, preferably jugularly, through superior vena cava SVC, right atrium RA and atrial septum AS up to the left atrium LA of the heart H. In a further alternative which is not illustrated the distal portion DP110b2 of the outer cannula CA110b is inserted further, for instance up to the ascending aorta AO.

After insertion of the outer cannula CA110b, e.g. after the outer cannula is in place, the distal portion DP110a of the bidirectional cannula CA110a is inserted through the proximal portion PP110b of the outer cannula CA110b, through the intermediate portion IP110b of the outer cannula CA110b, through the intermediate opening IO110b of the outer cannula CA110b, into the right atrium RA, through the right ventricle RV and at least to or up to the pulmonary artery PA.

Thereby, a first part FP-LP of the lumen portion of the outer cannula CA110b is formed between an inner surface of outer cannula CA110b and an outer surface of bidirectional cannula CA110a is formed. Furthermore, a second part SP-LP of the lumen portion of the outer cannula CA110b is formed between the intermediate opening IO110a of the bidirectional cannula CA110a and the distal portion DP110b of the outer cannula CA110b. The first part FP-LP of the lumen portion of the outer cannula CA110b may form a “dead” lumen portion.

At least one membrane pump MP10 may comprise for instance two blood ports which may be connected with the proximal end of the bidirectional cannula CA110a as is explained below in more detail. Membrane pump MP10 may be operated on an IABP console IABP10.

A connecting portion CP10 may be used, for instance a Y-connector. The connecting portion CP10 may be connected to:
a separated portion SP10a,
a separated portion SP10b, and
a separated portion SP10c.

Separated portion SP10a may connect the connecting portion with an inlet port of membrane pump MP11. A one-way valve V10a may be arranged within a separated portion SP10a. Alternatively and/or additionally a one-way valve may be arranged within separated portion SP10b.

Separated portion SP10b may connect an outlet port of device D10 with connecting portion CP10. Separated portion SP10c may be connected to proximal portion PP110a of bidirectional cannula.

A further separated portion SP10d may connect an outlet port of membrane pump MP10 with an inlet port of device D10.

There is the following function of arrangement 1000. Blood may be drained into the at least one distal opening DO110 of the bidirectional cannula CA110a in an aspiration phase from pulmonary artery PA, see arrow A110a. In an expulsion phase of the membrane pump operation blood is delivered out of the at least one intermediate opening IO110a of the bidirectional cannula CA110a and further through the at least one distal opening DO110b1 of the outer cannula CA11 Ob into left atrium LA in the first alternative (variant 1), see arrows A110d and A110e. In the second alternative (variant 2), blood is delivered out of the at least one intermediate opening IO110a of the bidirectional cannula CA110a and further through the at least one distal opening DO100b2 of the outer cannula CA11 Ob into left ventricle LV, see arrows Al10d and A110f.

Due to one-way valve V10a a fluid circulation is realized. Blood which is sucked into separated portion SP10c flows only through portion SP10a but not through portion SP10b, see arrow A110b. Device D10 may have an inherent valve function. Alternatively or additionally a further one-way valve may be used within separated portion SP10d or SP10c. Moreover, the one-way valves may also be integrated within the ports of membrane pump MP10. Blood which is expulsed out of membrane pump MP10 flows only through separated portions SP10d, device D10 and separated portion SP10b, see arrow A110c. One-way valve V10a blocks the flow through separated portion SP10a. Expulsed blood flows then through separated portion SP10c into bidirectional cannula CA110a.

The drained blood may be enriched in all alternatives with oxygen and/or it may be depleted from carbon dioxide outside of the body of a patient before it is delivered out of the intermediate opening IO110a of the bidirectional cannula CA110a. An ECCO₂R (extracorporeal carbon dioxide removal) device D10 may be used which may have lower pressures and/or throughput rates (volume per minute) compared to the usage of an oxygenator which may be used instead of the ECCO₂R device, especially within an ECMO (extracorporeal membrane oxygenation). However, both blood treatment methods are optionally.

With regard to the pumps see variants 3, 4, 6, i.e. FIGS. 3, 4 and 6. The proposed membrane pumps deliver a pulsatile blood flow. However, other pumps may also be used, for instance roller pumps (peristaltic pumps) or centrifugal pumps.

Furthermore, valves V9a to V10c or other sealing elements may be used, for instance multi-flap valves or another self-sealing member (for instance a simple sealing ring), i.e. for instance two flexible membranes. Other types of hemostasis valves may also be used.

Valve V9a, V10a may prevent that blood flows out of the proximal portion PP109b, PP110b of the outer cannula CA109b, CA110b, especially if the bidirectional cannula CA109a, Ca110a is not yet in the inserted state within outer cannula CA109b, CA110b. A multi-flap valve may be used for valve V9a, V10a.

Valve V9b, V10b may prevent that blood flows into the space or “dead” lumen between intermediate portion IP109b, IP110b and thus into a possible space between both cannulas CA109a, CA109b or CA110a, CA110b. This may result in preventing clotting of the blood in regions of the cannula system where the blood flow may be not high enough. A multi-flap valve may be used for valve V9b, V10b. Alternatively, a sealing ring or other sealing member may be used for valve V9b, V10b.

Valve V9c, V10c may be used to prevent that blood which is delivered out of intermediate opening IO109a, IO110a of bidirectional cannula CA109a, CA10a flows out of intermediate opening IO109b, IO110b of outer cannula CA109a, CA110b and thus in regions in which it should not be flow, i.e. the complete delivery flow may reach the distal opening DO109b, DO110b1 or DO110b2. If valve V9b, V10b is used valve V9c, V10c may be a simple sealing ring. However, other types of valves may also be used for valves V9c, V10c. A multi-flap valve may be used for valve V9c, V10c.

Valves V9b, V9c, V10b and V10c make sure that blood that flows out of intermediate opening IO109a, IO110a flows within outer cannula CA109b, CA110b to the distal opening DO109b, DO110b1 or DO110b2.

Although outer cannula CA110b is illustrated with different diameters, especially in the intermediate portion IP110b it is of course also possible to have a constant diameter along the longitudinal axis of outer cannula CA10b.

Furthermore, outer cannula CA110b may have a kink K in the intermediate portion. In a state without outer forces (base state) the kink K may include an angle in the range of 80 degrees to 130 degrees, preferably 110 degrees. The kink K may facilitate the insertion of the distal portion DP110a of bidirectional cannula CA110a through intermediate opening IO110b of outer cannula CA110b. However, it is also possible to use an outer cannula without a kink K. Intermediate opening IO110b of outer cannula CA110b may be arranged at kink K, preferably in a distance from the kink which is less than 3 cm or less than 2 cm or less than 1 cm.

There may be the following dimensions of cannula CA110a:

| Body height of | 110 | cm | 140 | cm | 160 | cm | 180 | cm | 200 cm |
|---|---|---|---|---|---|---|---|---|---|
| patient | | | | | | | | | |
| Distance | 8 cm | | 13 cm | | 18 cm | | 23 cm | | 28 cm |
| between IO110a | 6 cm to 10 cm | | 11 cm to 15 cm | | 16 cm to 20 cm | | 21 cm to 25 cm | | 24 cm to 30 cm |
| and DP110a | | | | | | | | | or 26 cm to 30 cm |
| Outer diameter, | 19 | Fr | 21 | Fr | 23 | Fr | 25 | Fr | 27 Fr or 29 Fr |
| for instance at | | | | | | | | | |
| PP110a and/or | | | | | | | | | |
| at DP110b | | | | | | | | | |

The distal portion DP110b of the outer cannula CA10b may have the same outer diameter as the distal portion DP110a of the bidirectional cannula CA110a. Alternatively, the distal portion DP110b of the outer cannula CA110b may have a greater outer diameter than the distal portion DP110a of the bidirectional cannula CA110a, for instance greater by at least 2 French but not greater than 8 French compared to the outer diameter of the distal portion DP110a of the bidirectional cannula CA110a. Alternatively, greater by at least 3 French but not greater than 8 French.

There may be the following dimensions of outer cannula CA110b in variant 1 (distal portion DP110b1 in LA):

| | | | | | |
|---|---|---|---|---|---|
| Body height of | 110 cm | 140 cm | 160 cm | 180 cm | 200 cm |
| patient | | | | | |
| Distance | 3 cm to 5 cm | 5 cm to 7 cm | 7 cm to 10 cm | 10 cm to 13 cm | 13 cm to 15 cm |
| between IO110b | | | | | |
| and DP110b1 | | | | | |
| Length of | 30 cm | 40 cm | 50 cm | 60 cm | 70 cm |
| cannula | (plus max. 5 cm | (plus max. 5 cm | (plus max. 5 cm | (plus max. 5 cm | (plus max. 5 cm |
| CA1010b | and/or minus | and/or minus | and/or minus | and/or minus | and/or minus |
| | max. 5 cm) | max. 5 cm) | max. 5 cm) | max. 5 cm) | max. 5 cm) |

There may be the following dimensions of outer cannula CA110b in variant 2 (distal portion DP110b2 in LV and/or in aorta AO):

| | | | | | |
|---|---|---|---|---|---|
| Body height of | 110 cm | 140 cm | 160 cm | 180 cm | 200 cm |
| patient | | | | | |
| Distance | 23 cm to 25 cm | 25 cm to 27 cm | 27 cm to 30 cm | 30 cm to 33 cm | 33 cm to 35 cm |
| between IO110b | | | | | |
| and DP110b2 | | | | | |
| Length of | 50 cm | 60 cm | 70 cm | 80 cm | 90 cm |
| cannula | (plus max. 5 cm | (plus max. 5 cm | (plus max. 5 cm | (plus max. 5 cm | (plus max. 5 cm |
| CA1010b | and/or minus | and/or minus | and/or minus | and/or minus | and/or minus |
| | max. 5 cm) | max. 5 cm) | max. 5 cm) | max. 5 cm) | max. 5 cm) |

The conical portion ConP may have a length of less than 10 cm for all cases, e.g. all body height. The conical portion ConP may have a reduction in diameter of at least 4 French of at least 5 French but preferably of not more than 8 French or of not more than 10 French. The conical portion ConP may be located distally adjacent to kink K and/or distally adjacent to intermediate portion IP110b.

Within the cannula system CS which comprises an inner cannula and an outer cannula there may also be the opposite flow directions compared to the flow directions mentioned above.

Furthermore, the inner cannula of the cannula system CS has not necessarily to be a bidirectional cannula, e.g. a unidirectional cannula may also be used, for instance a single lumen cannula without internal valve and without an internal valve function.

Other medical applications of the cannula system are possible as well.

In other words, the following is proposed:
a bi-directional flow catheter comprising for instance a two way valve. Access may be made via right jugular vein, for instance via right internal jugular vein rIJV, or left jugular vein, for instance left internal jugular vein IIJV, further to right atrium RA, then transseptal (atrial septum) into left atrium LA, through mitral valve MV, left ventricle LV at least to ascending aorta aAO or exactly to ascending aorta aAO.

Alternatively, a way or path through ventricle septum VS may be chosen,
right heart H assist, optionally combined with lung assist; the cannula is inserted preferably through vena cava, right atrium, right ventricle to pulmonary artery,
transcaval access is also possible for both possibilities, i.e. delivery into aorta AO or into pulmonary artery PA.

Variant 1, see FIG. 1: drainage of blood from left atrium LA and delivery of blood into aorta AO, preferably into ascending aorta aAO. Access may be made via atrial septum AS. The distance between distal tip and inlet/intermediate opening IO1 at intermediate portion IP1 of cannula CA1 may be between 10 and 25 cm (centimeter). A two-way directional valve may be placed at intermediate portion IP1.

Variant 2, see FIG. 2: drainage of blood from left ventricle LV and delivery of blood into aorta, preferably into ascending aorta aAO. Access may be made via atrial septum AS. The distance between the distal tip and the inlet/intermediate opening IO2 at intermediate portion IP2 of cannula CA2 may be between 5 and 12 cm. A two-way directional valve may be placed at intermediate portion IP2.

Variant 3, see FIG. 3: drainage of blood from right atrium RA and delivery of blood into aorta AO, preferably into ascending aorta aAO. Access may be made via atrial septum AS. At least one oxygenator OXY3 may be placed between catheter/cannula CA3 and membrane pump MP3. The distance between distal tip and inlet/intermediate opening IO3 at intermediate portion IP3 of cannula CA3 may be between 22 and 55 cm. A two-way directional valve may be placed at intermediate portion IP3.

Variant 4, see FIG. 4: drainage of blood from right atrium RA and delivery of blood into aorta AO, preferably into ascending aorta aAO. Access may be made transcaval, i.e. direct puncture transcaval from right atrium RA to ascending aorta aAO. At least one oxygenator OXY4 may be placed between catheter/cannula CA4 and membrane pump MP4. The distance between distal tip and inlet/intermediate opening IO4 at intermediate portion IP4 of cannula CA4 may be between 5 and 15 cm. A two way directional valve may be placed at intermediate portion IP4.

Variant 5, see FIG. 5: drain of blood from left ventricle LV and delivery of blood into ascending aorta aAO, preferably into ascending aorta aAO. Access may be made via ventricular septum VS. The distance between the distal tip and inlet/intermediate opening IO5 at intermediate portion IP5 of cannula CA5 may be between 10 and 25 cm. A two-way directional valve may be placed at intermediate portion IP5.

Variant 6, see FIG. 6: drainage of blood from right atrium RA and delivery of blood into aorta AO, preferably into ascending aorta aAO. Access may be made via ventricular septum VS. At least one oxygenator OXY6 may be placed between catheter/cannula CA and membrane pump MP. The distance between distal tip and inlet/intermediate opening IO6 at intermediate portion IP6 of cannula CA6 may be between 15 and 25 cm. A two-way directional valve may be placed at intermediate portion IP6.

The membrane pump that may be used in variants 1, 2 and 5 or in the other variants 3, 4 and 6 etc. may have only one port that is fluidly connected with the cannula, wherein the port does not comprise a valve. The cannula may include a two-way directional valve.

The membrane pump that may be used in variants 3, 4 and 6 or in the other variants 1, 2 and 5 etc. may have at least one inlet port and an outlet port in which a valve is mounted, respectively, for instance a one-way valve. Conduits that are connected to these ports may be united or joint between the pump and the cannula that is preferably a single lumen cannula. The cannula may include a two-way valve or alternatively several one way valves or another technical solution that enables a bidirectional flow in the proximal part of the cannula and direction dependent flows through the distal tip and through an intermediate opening of the catheter/cannula.

A pump having only one port may be used. Alternatively, a pump having an inlet port and an outlet port may be which comprise preferably one-way valves respectively. The two-way valve or the other fluidically mechanical solution within the cannula may still be used in order to control the direction of fluid flows through the distal part and through the at least one opening within the intermediate portion of the cannula.

The catheter/cannula diameter may be more than 23 F (French) and up to 36 F or more. No remaining room for blood flow in a vessel may be necessary anymore because the cannula may deliver sufficient flow rates alone, i.e. without the help of the blood circuit of body 100. For all embodiments, the outer diameter of the cannula/catheter may be equal to or more than 25 F up to 36 F, most preferred in the range of 29 F to 33 F. This may be more than 20 percent more compared to other solutions.

A dual chamber membrane pump with 40 ml (milliliter) or more and up to 160 ml pumping volume may be used. For all embodiments, a membrane pump, for instance MP3, MP4 and MP6 to MP10, with 60 ml or more up to 160 ml pumping volume may be used, most preferred in the range between 80 ml to 120 ml. This may be more than twice of the pumping volume that may be used for other solution.

At least one pump for driving a fluid flow may be used, for instance a membrane pump (pulsatile flow), especially comprising a flat membrane or a ring membrane. The pump volume of the pump may be preferably greater than the volume in the cannula between the distal end of the cannula and the inlet of the pump, especially a membrane pump, i.e. there may be no or only a small dead volume. This may result in no or only less clotting of blood within the cannula and/or the pump or variable volume reservoir.

The proposed solutions may be used for instance as:
as a short term solution for a bridge to decision, for instance up to 30 days or more, alternatively for instance up to 60 days depending on authorization,
a bridge to bridge (left ventricular assist device LVAD), for instance up to 30 days or more, alternatively for instance up to 60 days depending on authorization,
bridge to transplant, for instance up to 30 days or more, alternatively for instance up to 60 days depending on authorization,
a support in severe left ventricular failure,
during high-risk revascularization procedures, for instance on coronal arteries, and/or
right heart assist.

A connection to an IABP (Intra-Aortic Balloon Pump) console is possible, see for instance IABP consoles IABP7 to IABP10, i.e. at least one sensor for measuring the pulse or another signal of heart H may be used. Blood is delivered preferably in diastole of left ventricle, i.e. if heard does not pump out blood.

The proposed invention (see for instance any one of the FIGS. 1 to 10) may be applied for instance for treating:
acute myocardial infarction AMI (Heart Attack),
cardiogenic shock,
post cardiotomy patients, i.e. after treatment with heart lung machine,
OPCAB (Off Pump Coronary Artery Bypass)—as recovery support,
PCI (Percutaneous Cardiac intervention),
hypotension (Shock),
post heart transplantation, and
improve or enable extracorporeal membrane oxygenation (ECMO) weaning process.

There may be one or several of the following technical and/or medical effect(s):
pulsatile support and pumping synchronized with the heart,
increased circulatory blood flow,
safe, reliable and easy to use platform technology that may allow for instance the usage of an IABP (Intra-Aortic Balloon Pump) console,
fast, jugular percutaneous insertion, preferably in internal jugular vein,
unloading of the left ventricle,
increase of coronary and end-organ (liver or kidney for instance) perfusion,
low anticoagulation may be reached; anti-clotting time (ACT) may for instance be equal to or less than 180 seconds,
reduction in myocardial workload,
low complication rate, and
a pulsatile pump in combination with an oxygenator device may result in better cleaning or better wash out of the oxygenator device and may allow a longer usage of oxygenator.

A common insertion technique may be used:
only one lumen may be necessary, i.e. a single lumen cannula may be used, for instance 33 French cannula (11 mm),
short straightforward insertion,
up to 4 L/min (liter per minute) blood flow or more,
ECG (Electrocardiography) triggered pulsation,
driving by standard available IABP (Intra-Aortic Balloon Pump) consoles is possible. These consoles may already be there in many hospitals.

Furthermore, it is possible to use in all embodiments that are mentioned above an inner surface of the lumen portion that comprises a helically surface structure. The helically surface structure may have the effect that the fluid flow within the cannula is rotated as it moves through the cannula. Turbulences may be reduced thereby and/or it may be possible to reach much higher flow rates compared to cannulas that have a smooth inner surface, i.e. that do not have helical surface structures on their inner surfaces. However, it is of course possible to use cannulas without helical inner surface features, if for instance lower flow rates are necessary. The spirally turned flow and/or the rotated flow may prevent clotting of blood cells if the fluid flow comprises blood, especially in slow flow rate conditions. However, there may also be advantages if the fluid flow does not contain blood. The rotating flow may be a laminar flow.

Moreover, the cannula may be inserted endovascularly jugular and may be punctured from superior vena cava SVC or from right atrium RA transcaval to ascending aorta aAO. Alternatively, the cannula may be inserted endovascularly jugular through superior vena cava SVC and optionally into the right atrium RA and may be punctured from superior vena cava SVC or from right atrium RA transcaval to pulmonary artery PA.

In all embodiments a variable diameter arrangement may be used at the distal tip of the cannula, e.g. a cage arrangement or a balloon. It is possible to use a metal or another material than a metal for the cage arrangement, for instance a natural and/or biological material, especially cellulose, for instance cellulose that is treated to increase the hardness. Compatibility with body 100 and/or with blood may be improved thereby.

The bidirectional cannula may have only one end-hole, preferably in combination with a variable diameter arrangement around the single end-hole, for instance a cage arrangement.

The cannula(s) may be introduced jugular. This may allow usage of cannulas with greater outer diameter compared with femoral access. Furthermore, a cannula which is inserted jugular into heart H may be shorter than a cannula which is inserted femoral. Both aspects may have an influence to the pump, e.g. higher pumping volume may be possible etc.

The bidirectional cannula may have a length in the range of 40 cm (centimeters) to 50 cm, e.g. the length may be less than 80 cm and more preferably less than 60 cm. The outer diameter of the cannula(s) may be in the range of 19 Fr (French, 1 French equal to 0.33 mm (millimeter) or ⅓ mm) to 31 Fr, e.g. within the range of 21 Fr to 29 Fr. Higher flows, e.g. flows per minute, may be possible. This applies especially to the renal medical applications.

In all embodiments, optional one-way valves or other valves may be used within or at the distal end of the cannulas in addition to the one-way valves in the separated portions. These further one-way valves are backflow preventing valves which prevent that blood flows into outflow openings which are mainly used as outflow openings or that blood flows out of inflow openings which are mainly used as inflow openings. Alternatively and/or additionally, there may be one-way valves or other valves within an intermediated portions of the cannulas which have the same purpose. This is similar to flaps within the veins of the human body which flaps prevent backflow during the systole. These backflow preventing valves may be used in unidirectionally used cannula, especially in a single lumen cannula and/or in a dual lumen cannula as mentioned above.

In all embodiments one of the following methods may be used to bring or guide a guide wire and/or a catheter around or along the acute angle within the left ventricle LV, see for instance FIGS. 1 to 6 and FIG. 10. At least one snare may be used to catch the catheter and/or the guide wire in the left ventricle LV. The methods may be performed independent whether there is jugular access or a femoral access or another access for the catheter and/or the guide wire.

Variant A (catching the catheter with the snare):
1) Introducing a catheter through the right atrium RA, the atrial septum AS (a puncturing step may be performed earlier or using the catheter, e.g. using a needle and/or RF (radio frequency) tip/wire within the catheter). The catheter may be introduced further through the hole (puncture) in the atrial septum AS through left atrium LA, through mitral valve MV into the left ventricle LV.
2) Introducing a snare from descending aorta AO through aortic valve AV into left ventricle LV. This step may be performed also before step 1.
3) Catching the catheter in the left ventricle LV using the snare.
4) Pulling the snare and the distal end of the catheter therewith to the aorta AO.
5) Introducing a guide wire through the catheter.
6) Forwarding the guide wire out of the distal end of the catheter. Slight loosening of the snare may be optionally performed thereby.
7) As the guide wire is already within the snare, pull back the snare to a region in which only the guide wire is located but not the catheter.
8) Fix the guide wire using the snare, e.g. contract the snare and/or tighten the snare.
9) Optional, externalizing for instance the distal end of the guide wire out of the body. This step is optionally, because the proximal end of the snare is already outside of the body.
10) Remove catheter, e.g. pull back the catheter.
11) Introduce cannula using the guide wire, e.g. pushing the cannula along and/or over the guide wire until it is on its final place.

Variant B (catching the guide wire with the snare):
1) Introducing a catheter through the right atrium RA, through the atrial septum AS (a puncturing step may be performed earlier or through catheter, use needle and/or RF (radio frequency) tip/wire).

Introducing the catheter further through left atrium LA, mitral valve MV into the left ventricle LV.
2) Introducing a guide wire through the catheter until the distal end of the guide wire comes out of the distal end of the catheter within the left ventricle LV. The RF wire may be used also as a guide wire.
3) Introducing a snare from descending aorta AO through aortic valve AV into left ventricle LV. This step may be performed before step 1 and/or before step 2.
3) Catching the distal end of the guide wire in the left ventricle LV using the snare.
4) Fixation of the guide wire using the snare.
5) Pulling the snare and the distal end of the guide wire therewith to the aorta AO.
6) Optional, externalizing guide wire by pulling it out of the body using the snare. This step is optional as the snare is already outside of the body from where it has been introduced.
7) Remove catheter, e.g. by pulling it back along the guide wire.
8) Introduce cannula over/along the guide wire until it is on place.

The following method may also be used in all corresponding embodiments for introducing a cannula jugularly transseptally:
1) Introduce a first snare into an internal jugular vein IN, for instance into the right jugular vein RJV or into the left jugular vein LJV.
2) Advancing the first snare to inferior vena cava IVC.
3) Introducing a catheter into a common femoral vein CFV (left or right).
4) Advancing the catheter through the first snare into an inferior vena cava IVC.
5) Advancing the catheter through the first snare into the vena cava VC in an antegrade fashion.
6) Advancing the catheter through the first snare into the right atrium RA in an antegrade fashion.
7) Advancing the catheter through the first snare and from the right atrium RA transseptally through the atrial septum into the left atrium LA in an antegrade fashion. Puncturing of atrial septum may have been performed earlier. Alternatively, the catheter is used to puncture the atrial septum, for instance using a needle or using a RF (radio frequency) wire/tip which is introduced trough the catheter.
8) Advancing the catheter through the first snare and advancing the catheter across the mitral valve MV and into the left ventricular outflow tract, e.g. the left ventricle LV.
9) Advancing a second snare in the ascending aorta AO catching and snaring a distal portion of the catheter (Variant A) within the left ventricle LV. The second snare may optionally be introduced through an artery, which may include, but is not limited to, a radial artery, a brachial artery, an axillary artery, a subclavian artery, a carotid artery, or common femoral artery, and advanced retrograde into the aorta AO and into the left ventricle LV. The second snare may be already introduced before the catheter is introduced. Alternatively, a guide wire may be inserted into the catheter until a distal end of the guide wire comes out of a distal opening of the catheter. This distal end of the guide wire is then caught and snared within the left ventricle (Variant B)
10) Pulling the catheter (Variant A) or the guide wire (Variant B) into the aorta AO in an antegrade fashion using the second snare.
11) In variant A, advancing a guide wire through the catheter and through the first snare in antegrade fashion to the ascending aorta AO and through the second snare. Snaring the distal end of the guide wire in variant A but not the catheter.
12) In both variants A and B remove the catheter with the guide wire remaining in the heart H and through the first snare after the catheter is removed.
13) Externalizing a proximal portion of the guide wire from femoral vein, through inferior vena cava IVC, through inferior vena cava SVC, into the internal jugular vein IN and then out of the internal jugular vein IN using the first snare, for instance left jugular vein LJV or right jugular vein RJV. In some embodiments the snare may externalize a different portion of the guide wire, for instance an intermediate portion.
14) Advancing a cannula using the guide wire and/or along and/or over the guide wire from the internal jugular vein IJV. The cannula may be any of the cannulas described in this specification or known in the art. Especially, an outer cannula may be advanced over the guide wire from the internal jugular vein IJV. An inner cannula may optionally be advanced through a port proximal of the distal end of the outer cannula. The inner cannula and the outer cannula may be positioned as described in this description, or if a single multi-lumen cannula is used, it may be positioned in a similar manner.
15) Optionally, a distal portion of the guide wire may be externalized out of the body through the artery.

This step is optional because the second snare is already externalized and may form a secure anchor for the distal portion of the guide wire.

Subclavian arteries/veins or other arteries/veins may be used for introducing the snare(s) because the snares require smaller diameters, e.g. less than 10 French (1 French equal to ⅓ mm (millimeter)) or less than 8 French, e.g. more than 3 French, compared to the diameters of the cannula(s).

In the following details of a method for puncturing transseptally through the atrial septum AS or the ventricular septum VS of the heart H or of other tissue are provided. However, other methods may be used as well, for instance using a needle.

A catheter and/or a wire may be used which has a distal tip which can be heated, for instance using RF (radio frequency) energy, alternating current (ac), direct current (dc) etc. Thus, e.g. a hole may be burned into the septum, e.g. the atrial septum AS, during puncturing, for instance using temperatures above 100° C. (degrees Celsius) or above 200° C., less than 1000° C. for instance.

The RF (radio frequency) may be in the range of 100 kHz (kilohertz) to 1 MHz (Megahertz) or in the range of 300 kHz to 600 kHz, for instance around 500 MHz, i.e. in the range of 450 kHz to 550 kHz, e.g. 468 kHz.

The power of the radio frequency energy may have a maximum of 50 Watt. A power range of 5 W (watt) to 100 W may be used, for instance a range of 10 W to 50 W.

A sinus current/voltage may be used for the RF. The sinus current/voltage may be continuous. Alternatively, a pulsed sinus current/voltage may be used for the RF.

All parameters or some of the parameters of the RF equipment may be adjustable by an operator who performs the puncturing, for instance dependent on the specifics of the septum, e.g. normal septum, fibrotic septum, aneurysmal septum, etc. Preferably, the power may be adjustable.

A solution of Baylis Medical (may be a trademark), Montreal, Canada may be used, for instance NRG® trans-septal needle or Supra Cross® RF Wire technology. RF generator of type RFP-100A or a further development of this model may be used. This RF generator uses for example a frequency of 468 kHz (kilohertz).

A single puncture of the septum AS, VS or of other tissue may be performed from a jugular access or from a femoral access or from another appropriate access using the RF energy. Smaller angles may be possible for guiding the catheter if for instance compared with a needle.

Alternatively, the RF method may be used also if two separate punctures are made in the septum. However, usage of needles is possible as well. One of the punctures using the RF method may be made through left jugular vein LJV and the other puncture of the atrial septum AS may be made through the right jugular vein RJV.

It is possible to introduce both guide wires first through the atrial septum AS. Preferably, separate holes are used for each of the guide wires. Guide wire(s) may be used which include an RF tip. Alternatively, the wire(s) having the RF tip may be pulled back and a further wire may be introduced through the catheter.

Only after both guide wires are in place, both cannulas may be introduced using a respective one of the guide wires.

Alternatively, the first puncture may be performed using RF energy or a needle. Thereafter, the first cannula for blood transfer is inserted using the first guide wire. After insertion of the first cannula, the second puncture may be made. A second guide wire or the first guide wire may be used to introduce the second cannula.

Puncturing of the atrial septum AS or of ventricular septum VS or of other tissue, for instance for transcaval access, may be assisted by at least one medical imaging method, preferably by at least two medical imaging methods.

US (ultra-sonic) echo imaging may be used to visualize the movement of heart H and the location of the valves of heart H. No dangerous radiation may result from ultra-sonic imaging. An ultra-sonic transmitter may be introduced for instance via the esophagus, e.g. trans esophagus echo (TEE) may be used.

X-ray radiation preferably in combination with fluorescence (fluoroscopy), may be used in order to visualize the location of catheters (comprising for instance at least one X-ray marker, or the devises are usually radiopaque) and/or the location of guide wire(s), snares etc.

Thus, transseptal puncturing or puncturing of other tissue may be guided by TEE and by fluoroscopy or by other imaging methods. At least two different image generating methods may be used.

In all embodiments mentioned above, it is also possible to use a soft guide wire and a stiffer guide wire which does not bend so easy if compared with the soft guide wire. The following steps may be performed, preferably in combination with snaring:
1) Introduce a soft guide wire.
2) Introduce catheter using the soft wire as a guide.
3) Optionally, remove soft wire, for instance by pulling back the soft wire out of the catheter.
4) Introduce stiffer guide wire into the catheter, e.g. there may be a change of wire from soft wire to the stiffer wire.

The catheter may be removed, e.g. pulled back. Thereafter, the stiffer wire may be used to introduce a cannula or cannulas.

Although embodiments of the present disclosure and their advantages have been described in detail, it should be understood that various changes, substitutions and alterations may be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. Further, it is possible to combine embodiments mentioned in the first part of the description with examples of the second part of the description which relates to FIGS. 1 to 10.

1. Cannula (CA1 to CA7, CA107, CA108, CA109a, CA110a) for endovascular and/or jugular blood circuit support, comprising:
a proximal portion (PP1 to PP6),
a distal portion (DP1 to DP7) that comprises at least one distal opening (DO1 to DO7),
a lumen portion (LP) that extends from the proximal portion (PP1 to PP6) to the at least one distal opening (DO1 to DO7), and
at least one intermediate portion (IP1 to IP7) that is arranged between the proximal portion (PP1 to PP6) and the distal portion (DP1 to DP7),
wherein the intermediate portion (IP1 to IP7) comprises at least one intermediate opening (IO1 to IO7),
wherein the intermediate portion (IP1 to IP7) is configured such that more than 90 volume percent of the fluid flow are drained from the intermediate opening (IO1 to IO7) if a fluid flow within the proximal portion (PP1 to PP6) is directed proximally and such that more than 90 volume percent of the fluid flow are delivered through the at least one distal opening (DO1 to DO7) if a fluid flow within the proximal portion (PP1 to PP6) is directed distally, or
b) the intermediate portion (IP109a, IP110a) is configured such that more than 90 volume percent of the fluid flow are drained from the at least one distal opening (DO109a, DO110a) if a fluid flow within the proximal portion (PP109a, PP110a) is directed proximally and such that more than 90 volume percent of the fluid flow are delivered through the intermediate opening (IO109a, IO110a) if a fluid flow within the proximal portion (PP109a, PP110a) is directed distally.

2. Cannula (CA1 to CA7) according to claim 1, wherein the cannula (CA1 to CA7) has one of the following dimensions:
a1) a distance between a distal end of the cannula (CA1) and the at least one intermediate opening (IO1) is in the range of 10 cm to 25 cm and a total length of the cannula (CA1) is in the range of 55 cm to 85 cm, preferably 65 cm,
wherein the cannula (CA1) is adapted to be inserted endovascularly, preferably jugular, through vena cava (VC), right atrium (RA), atrial septum (AS), left atrium (LA), left ventricle (LV) at least into aorta (AO) with blood drainage from the left atrium (LA) and with blood delivery into the aorta (AO),
a2) a distance between a distal end of the cannula (CA2) and the at least one intermediate opening (IO2) is in the range of 5 cm and 12 cm and a total length of the cannula (CA2) is in the range of 55 cm to 85 cm, preferably 65 cm,
wherein the cannula (CA2) is adapted to be inserted endovascularly, preferably jugular, through vena cava (VC), right atrium (RA), atrial septum (AS), left atrium (LA), left ventricle (LV) at least into aorta (AO) with blood drainage from the left ventricle (LV) and with blood delivery into the aorta (AO),
a3) a distance between a distal end of the cannula (CA3) and an intermediate opening (IO3) is in the range of 22 cm to 35 cm and a total length of cannula (CA3) is in the range of 55 cm to 85 cm, preferably 65 cm, wherein the cannula (CA3) is adapted to be inserted endovascularly, preferably jugular, through vena cava (VC), right atrium (RA), atrial septum (AS), left atrium (LA), left ventricle (LV) at least into aorta (AO) with blood drainage from the right atrium (RA) and with blood delivery into the aorta (AO),
a3a) a distance between a distal end of the cannula and the at least one intermediate opening is in the range of 27 cm to 40 cm and a total length of cannula is in the range of 55 cm to 85 cm, preferably 65 cm, wherein the cannula is adapted to be inserted endovascularly, preferably jugular, through vena cava (VC), right atrium (RA), atrial septum (AS), left atrium (LA), left ventricle (LV) at least into aorta (AO) with blood drainage from the vena cava (VC) and with blood delivery into the aorta (AO),
a4) a distance between a distal end of the cannula (CA4) and the at least one intermediate opening (IO4) is in the range of 5 cm to 15 cm and a total length of cannula (CA4) is in the range of 45 cm to 65 cm, preferably 55 cm,
wherein the cannula (CA4) is adapted to be inserted endovascularly, preferably jugular, through vena cava (VC), right atrium (RA) and punctured transcaval from right atrium (RA) to aorta (AO) with blood drainage from the right atrium (RA) and with blood delivery into the aorta (AO), or
a distance between a distal end of the cannula and the at least one intermediate opening is in the range of 10 cm to 20 cm and a total length of cannula (CA4) is in the range of 45 cm to 65 cm, preferably 55 cm, wherein the cannula (CA4) is adapted to be inserted endovascularly, preferably jugular, through vena cava (VC), right atrium (RA) and punctured transcaval from right atrium (RA) to aorta (AO) with blood drainage from the vena cava (VC) and with blood delivery into the aorta (AO),
a4a) a distance between a distal end of the cannula (CA4a) and the at least one intermediate opening (IO4a) is in the range of 10 cm to 25 cm and a total length of cannula (CA4a) is in the range of 45 cm to 65 cm, preferably 55 cm,
wherein the cannula (CA4a) is adapted to be inserted endovascularly, preferably jugular, through vena cava (VC) and punctured transcaval from the vena cava (VC) to aorta (AO) with blood drainage from the vena cava (VC) and with blood delivery into the aorta (AO),
a5) a distance between a distal end of the cannula (CA5) and the at least one intermediate opening (IO5) is in the range of 10 cm to 25 cm and a total length of cannula (CA5) is in the range of 55 cm to 85 cm, preferably 65 cm,
wherein the cannula (CA5) is adapted to be inserted endovascularly, preferably jugular, through vena cava (VC), right atrium (RA), right ventricle (RV), ventricle septum (VS), left ventricle at least to aorta (AO) with blood drainage from the left ventricle (LV) and with blood delivery into the aorta (AO),
a6) a distance between a distal end of the cannula (CA6) and the at least one intermediate opening (IO6) is in the range of 15 cm to 25 cm or in the range of 10 cm to 25 cm and a total length of cannula (CA6) is in the range of 55 cm and 85 cm, preferably 65 cm,
wherein the cannula (CA6) is adapted to be inserted endovascularly, preferably jugular, through vena cava (VC), right atrium (RA), right ventricle (RV), ventricle septum (VS), left ventricle (LV) at least to aorta (AO) with blood drainage from the right atrium (RA) and with blood delivery into the aorta (AO), or a distance between a distal end of the cannula (CA6) and the at least one intermediate opening (IO6) is in the range of 10 cm to 25 cm and a total length of cannula (CA6) is in the range of 55 cm and 85 cm, preferably 65 cm,
wherein the cannula (CA6) is adapted to be inserted endovascularly, preferably jugular, through vena cava (VC), right atrium (RA), right ventricle (RV), ventricle septum (VS), left ventricle (LV) at least to aorta (AO) with blood drainage from the right ventricle (RV) and with blood delivery into the aorta (AO), or a distance between a distal end of the cannula (CA6) and the at least one intermediate opening (IO6) is in the range of 20 to 40 cm and a total length of cannula (CA6) is in the range of 55 cm and 85 cm, preferably 65 cm,
wherein the cannula (CA6) is adapted to be inserted endovascularly, preferably jugular, through vena cava (VC), right atrium (RA), right ventricle (RV), ventricle septum (VS), left ventricle (LV) at least to aorta (AO) with blood drainage from vena cava (VC) and with blood delivery into the aorta (AO),
a7) a distance between a distal end of the cannula (CA7) and the at least one intermediate opening (IO7) is in the range of 15 cm to 25 cm and a total length of the cannula (CA7) in the range of 55 cm to 85 cm, preferably 65 cm,
wherein the cannula (CA7) is adapted to be inserted endovascularly, preferably jugular, through vena cava (VC), right atrium (RA), right ventricle (RV) at least to pulmonary artery (PA) with blood drainage from the right atrium (RA) and with blood delivery into the pulmonary artery (PA),
a8) a distance between a distal end of the cannula and the at least one intermediate opening is in the range of 10 cm and 20 cm and a total length of the cannula is in the range of 55 cm to 85 cm, preferably 65 cm, wherein the cannula is adapted to be inserted endovascularly, preferably jugular, through vena cava (VC), right atrium (RA), right ventricle (RV) at least to pulmonary artery (PA) with blood drainage from the right ventricle (RV) and with blood delivery into the pulmonary artery (PA),
a9) a distance between a distal end of the cannula and the at least one intermediate opening is in the range of 25 cm and 35 cm and a total length of cannula is in the range of 55 cm to 85 cm, preferably 65 cm, wherein the cannula is adapted to be inserted endovascularly, preferably jugular, through vena cava (VC), right atrium (RA), right ventricle (RV) at least to pulmonary artery (PA) with blood drainage from the vena cava (VC) and with blood delivery into the pulmonary artery (PA),
a10) a distance between a distal end of the cannula (CA10) and the at least one intermediate opening (IO10) is in the range of 5 cm to 15 cm and a total length of cannula (CA10) is in the range of 45 cm to 65 cm, preferably 55 cm,
wherein the cannula (CA10) is adapted to be inserted endovascularly, preferably jugular, through vena cava (VC), right atrium (RA) and punctured transcaval from the right atrium (RA) to pulmonary artery (PA) with blood drainage from the right atrium (RA) and with blood delivery into the pulmonary artery (PA), or
a distance between a distal end of the cannula and the at least one intermediate opening is in the range of 10 cm to 20 cm and a total length of cannula is in the range of 45 cm to 65 cm, preferably 55 cm, wherein the cannula is adapted to be inserted endovascularly, preferably jugular, through vena cava (VC), right atrium (RA) and punctured transcaval from the right atrium (RA) to pulmonary artery (PA) with blood drainage from the vena cava (VC) and with blood delivery into the pulmonary artery (PA),
a10a) a distance between a distal end of the cannula (CA10a) and the at least one intermediate opening (IO10a) is in the range of 10 cm to 20 cm and a total length of cannula (CA10a) is in the range of 45 cm to 65 cm, preferably 55 cm,
wherein the cannula (CA10a) is adapted to be inserted endovascularly, preferably jugular, through vena cava (VC) and punctured transcaval from the vena cava (VC) to pulmonary artery (PA) with blood drainage from the vena cava (VC) and with blood delivery into the pulmonary artery (PA),
a11a) a distance between a distal end of the cannula (CA107) and the at least one intermediate opening (IO107) is in the range of 5 cm to 30 cm and a total length of cannula (CA107) is in the range of 45 cm to 65 cm, preferably 55 cm,
wherein the cannula (CA107, CA108) is adapted to be inserted endovascularly, preferably jugular, through superior vena cava (VC), through right atrium (RA) and inferior vena cava (IVC) with blood drainage from at least one renal vein (rV1, rV2) or from both renal veins (rV1, rV2) and with blood delivery into the right atrium (RA).

3. Cannula (CA1 to CA7) according to claim 2, wherein the maximal outer diameter of the cannula (CA1 to CA7) is in the range of 25 F to 36 F or preferably in the range of 29 F to 33 F.

4. Cannula (CA1 to CA7) according to claim 1, wherein the cannula (CA1 to CA7) has one of the following dimensions:
a1) a distance between a distal end of the cannula (CA1) and the at least one intermediate opening (IO1) is in the range of 10 cm to 25 cm,
wherein the cannula (CA1) is adapted to be inserted endovascularly, preferably jugular, through vena cava (VC), right atrium (RA), atrial septum (AS), left atrium (LA), left ventricle (LV) at least into aorta (AO) with blood drainage from the left atrium (LA) and with blood delivery into the aorta (AO),
a2) a distance between a distal end of the cannula (CA2) and the at least one intermediate opening (IO2) is in the range of 5 cm and 12 cm,
wherein the cannula (CA2) is adapted to be inserted endovascularly, preferably jugular, through vena cava (VC), right atrium (RA), atrial septum (AS), left atrium (LA), left ventricle (LV) at least into aorta (AO) with blood drainage from the left ventricle (LV) and with blood delivery into the aorta (AO),
a3) a distance between a distal end of the cannula (CA3) and the at least one intermediate opening (IO3) is in the range of 22 cm to 35 cm,
wherein the cannula (CA3) is adapted to be inserted endovascularly, preferably jugular, through vena cava (VC), right atrium (RA), atrial septum (AS), left atrium (LA), left ventricle (LV) at least into aorta (AO) with blood drainage from the right atrium (RA) and with blood delivery into the aorta (AO), a3a) a distance between a distal end of the cannula and the at least one intermediate opening is in the range of 27 cm to 40 cm,
wherein the cannula is adapted to be inserted endovascularly, preferably jugular, through vena cava (VC), right atrium (RA), atrial septum (AS), left atrium (LA), left ventricle (LV) at least into aorta (AO) with blood drainage from the vena cava (VC) and with blood delivery into the aorta (AO),
a4) a distance between a distal end of the cannula (CA4) and the at least one intermediate opening (IO4) is in the range of 5 cm to 15 cm,
wherein the cannula (CA4) is adapted to be inserted endovascularly, preferably jugular, through vena cava (VC), right atrium (RA) and punctured transcaval from the right atrium (RA) to aorta (AO) with blood drainage from the right atrium (RA) and with blood delivery into the aorta (AO), or
a distance between a distal end of the cannula and the at least one intermediate opening is in the range of 10 cm to 20 cm,
wherein the cannula (CA4) is adapted to be inserted endovascularly, preferably jugular, through vena cava (VC), right atrium (RA) and punctured transcaval from right atrium (RA) to aorta (AO) with blood drainage from the vena cava (VC) and with blood delivery into the aorta (AO),
a4a) a distance between a distal end of the cannula (CA4a) and the at least one intermediate opening (IO4a) is in the range of 10 cm to 25 cm,
wherein the cannula (CA4a) is adapted to be inserted endovascularly, preferably jugular, through vena cava (VC) and punctured transcaval from the vena cava (VC) to aorta (AO) with blood drainage from the vena cava (VC) and with blood delivery into the aorta (AO),
a5) a distance between a distal end of the cannula (CA5) and the at least one intermediate opening (IO5) is in the range of 10 cm to 25 cm,
wherein the cannula (CA5) is adapted to be inserted endovascularly, preferably jugular, through vena cava (VC), right atrium (RA), right ventricle (RV), ventricle septum (VS), left ventricle at least to aorta (AO) with blood drainage from the left ventricle (LV) and with blood delivery into the aorta (AO),
a6) a distance between a distal end of the cannula (CA6) and the at least one intermediate opening (IO6) is in the range of 15 cm to 25 cm,
wherein the cannula (CA6) is adapted to be inserted endovascularly, preferably jugular, through vena cava (VC), right atrium (RA), right ventricle (RV), ventricle septum (VS), left ventricle (LV) at least to aorta (AO) with blood drainage from the right atrium (RA) and with blood delivery into the aorta (AO), or a distance between a distal end of the cannula (CA6) and the at least one intermediate opening (IO6) is in the range of 10 cm to 20 cm,
wherein the cannula (CA6) is adapted to be inserted endovascularly, preferably jugular, through vena cava (VC), right atrium (RA), right ventricle (RV), ventricle septum (VS), left ventricle (LV) at least to aorta (AO) with blood drainage from the right ventricle (RV) and with blood delivery into the aorta (AO), or a distance between a distal end of the cannula (CA6) and the at least one intermediate opening (IO6) is in the range of 20 to 40 cm,
wherein the cannula (CA6) is adapted to be inserted endovascularly, preferably jugular, through vena cava (VC), right atrium (RA), right ventricle (RV), ventricle septum (VS), left ventricle (LV) at least to aorta (AO) with blood drainage from vena cava (VC) and with blood delivery into the aorta (AO),
a7) a distance between a distal end of the cannula (CA7) and the at least one intermediate opening (IO7) is in the range of 15 cm to 25 cm,
wherein the cannula (CA7) is adapted to be inserted endovascularly, preferably jugular, through vena cava (VC), right atrium (RA), right ventricle (RV) at least to pulmonary artery (PA) with blood drainage from the right atrium (RA) and with blood delivery into the pulmonary artery (PA),
a8) a distance between a distal end of the cannula and the at least one intermediate opening is in the range of 10 cm and 20 cm,
wherein the cannula (CA7) is adapted to be inserted endovascularly, preferably jugular, through vena cava (VC), right atrium (RA), right ventricle (RV) at least to pulmonary artery (PA) with blood drainage from the right ventricle (RV) and with blood delivery into the pulmonary artery (PA),
a9) a distance between a distal end of the cannula and the at least one intermediate opening is in the range of 25 cm and 35 cm,
wherein the cannula (CA7) is adapted to be inserted endovascularly, preferably jugular, through vena cava (VC), right atrium (RA), right ventricle (RV) at least to pulmonary artery (PA) with blood drainage from the vena cava (VC) and with blood delivery into the pulmonary artery (PA),
a10) a distance between a distal end of the cannula (CA10) and the at least one intermediate opening (IO10) is in the range of 5 cm to 15 cm,
wherein the cannula (CA10) is adapted to be inserted endovascularly, preferably jugular, through vena cava (VC), right atrium (RA) and punctured transcaval from the right atrium (RA) to the pulmonary artery (PA) with blood drainage from the right atrium (RA) and with blood delivery into the pulmonary artery (PA), or
a distance between a distal end of the cannula and the at least one intermediate opening is in the range of 10 cm to 20 cm,
wherein the cannula is adapted to be inserted endovascularly, preferably jugular, through vena cava (VC), right atrium (RA) and punctured transcaval from the right atrium (RA) to pulmonary artery (PA) with blood drainage from the vena cava (VC) and with blood delivery into the pulmonary artery (PA),
a10a) a distance between a distal end of the cannula (CA10a) and the at least one intermediate opening (IO10a) is in the range of 10 cm to 20 cm,
wherein the cannula (CA10a) is adapted to be inserted endovascularly, preferably jugular, through vena cava (VC) and punctured transcaval from vena cava (VC) to pulmonary artery (PA) with blood drainage from the vena cava (VC) and with blood delivery into the pulmonary artery (PA),
a1l a) a distance between a distal end of the cannula (CA107) and the at least one intermediate opening (IO107) is in the range of 5 cm to 30 cm,
wherein the cannula (CA107, CA108) is adapted to be inserted endovascularly, preferably jugular, through superior vena cava (VC), through right atrium (RA) and inferior vena cava (IVC) with blood drainage from at least one renal vein (rV1, rV2) or from both renal veins (rV1, rV2) and with blood delivery into the right atrium (RA).

5. Cannula (CA1 to CA7) according to claim 4, wherein the maximal outer diameter of the cannula (CA1 to CA7) is in the range of 25 F to 36 F or preferably in the range of 29 F to 33 F.

6. Cannula (CA1 to CA7) according to claim 1, wherein the cannula (CA1 to CA7) has one of the following dimensions:
a1) a total length of cannula (CA1) is in the range of 55 cm to 85 cm, preferably 65 cm,
wherein the cannula (CA1) is adapted to be inserted endovascularly, preferably jugular, through vena cava (VC), right atrium (RA), atrial septum (AS), left atrium (LA), left ventricle (LV) at least into aorta (AO) with blood drainage from the left atrium (LA) and with blood delivery into the aorta (AO),
a2) a total length of cannula (CA2) is in the range of 55 cm to 85 cm, preferably 65 cm,
wherein the cannula (CA2) is adapted to be inserted endovascularly, preferably jugular, through vena cava (VC), right atrium (RA), atrial septum (AS), left atrium (LA), left ventricle (LV) at least into aorta (AO) with blood drainage from the left ventricle (LV) and with blood delivery into the aorta (AO),
a3) a total length of cannula (CA3) is in the range of 55 cm to 85 cm, preferably 65 cm,
wherein the cannula (CA3) is adapted to be inserted endovascularly, preferably jugular, through vena cava (VC), right atrium (RA), atrial septum (AS), left atrium (LA), left ventricle (LV) at least into aorta (AO) with blood drainage from the right atrium (RA) and with blood delivery into the aorta (AO),
a3a) a total length of cannula is in the range of 55 cm to 85 cm, preferably 65 cm,
wherein the cannula is adapted to be inserted endovascularly, preferably jugular, through vena cava (VC), right atrium (RA), atrial septum (AS), left atrium (LA), left ventricle (LV) at least into aorta (AO) with blood drainage from the vena cava (VC) and with blood delivery into the aorta (AO),
a4) a total length of cannula (CA4) is in the range of 45 cm to 65 cm, preferably 55 cm,
wherein the cannula (CA4) is adapted to be inserted endovascularly, preferably jugular, through vena cava (VC), right atrium (RA) and punctured transcaval from the right atrium (RA) to aorta (AO) with blood drainage from the right atrium (RA) and with blood delivery into the aorta (AO),
a total length of cannula (CA4) is in the range of 45 cm to 65 cm, preferably 55 cm,
wherein the cannula (CA4) is adapted to be inserted endovascularly, preferably jugular, through vena cava (VC), right atrium (RA) and punctured transcaval from right atrium (RA) to aorta (AO) with blood drainage from the vena cava (VC) and with blood delivery into the aorta (AO),
a4a) a total length of cannula (CA4a) is in the range of 45 cm to 65 cm, preferably 55 cm,
wherein the cannula (CA4a) is adapted to be inserted endovascularly, preferably jugular, through vena cava (VC) and punctured transcaval from the vena cava (VC) to aorta (AO) with blood drainage from the vena cava (VC) and with blood delivery into the aorta (AO),
a5) a total length of cannula (CA5) in the range of 55 cm to 85 cm, preferably 65 cm,
wherein the cannula (CA5) is adapted to be inserted endovascularly, preferably jugular, through vena cava (VC), right atrium (RA), right ventricle (RV), ventricle septum (VS), left ventricle (LV) at least to aorta (AO) with blood drainage from the left ventricle (LV) and with blood delivery into the aorta (AO),
a6) a total length of cannula (CA6) is in the range of 55 cm and 85 cm, preferably 65 cm,
wherein the cannula (CA6) is adapted to be inserted endovascularly, preferably jugular, through vena cava (VC), right atrium (RA), right ventricle (RV), ventricle septum (VS), left ventricle (LV) at least to aorta (AO) with blood drainage from the right atrium (RA) or from the right ventricle (RV) or from the vena cava (VC) and with blood delivery into the aorta (AO),
a7) a total length of cannula (CA7) in the range of 55 cm to 85 cm, preferably 65 cm,
wherein the cannula (CA7) is adapted to be inserted endovascularly, preferably jugular, through vena cava (VC), right atrium (RA), right ventricle (RV) at least to pulmonary artery (PA) with blood drainage from the right atrium (RA) and with blood delivery into the pulmonary artery (PA),
a8) a total length of cannula is in the range of 55 cm to 85 cm, preferably 65 cm,
wherein the cannula is adapted to be inserted endovascularly, preferably jugular, through vena cava (VC),
right atrium (RA), right ventricle (RV) at least to pulmonary artery (PA) with blood drainage from the right ventricle (RV) and with blood delivery into the pulmonary artery (PA),
a9) a total length of cannula is in the range of 55 cm to 85 cm, preferably 65 cm,
wherein the cannula is adapted to be inserted endovascularly, preferably jugular, through vena cava (VC), right atrium (RA), right ventricle (RV) at least to pulmonary artery (PA) with blood drainage from the vena cava (VC) and with blood delivery into the pulmonary artery (PA),
a10) a total length of cannula (CA10) is in the range of 45 cm to 65 cm, preferably 55 cm,
wherein the cannula (CA10) is adapted to be inserted endovascularly, preferably jugular, through vena cava (VC), right atrium (RA) and punctured transcaval from the right atrium (RA) to the pulmonary artery (PA) with blood drainage from the right atrium (RA) and with blood delivery into the pulmonary artery (PA), or
a total length of cannula is in the range of 45 cm to 65 cm, preferably 55 cm,
wherein the cannula is adapted to be inserted endovascularly, preferably jugular, through vena cava (VC), right atrium (RA) and punctured transcaval from the right atrium (RA) to pulmonary artery (PA) with blood drainage from the vena cava (VC) and with blood delivery into the pulmonary artery (PA),
a10a) a total length of cannula (CA10a) is in the range of 45 cm to 65 cm, preferably 55 cm, wherein the cannula (CA10a) is adapted to be inserted endovascularly, preferably jugular, through vena cava (VC) and punctured transcaval from vena cava (VC) to pulmonary artery (PA) with blood drainage from the vena cava (VC) and with blood delivery into the pulmonary artery (PA),
a11a) a total length of cannula (CA107) is in the range of 35 cm to 65 cm, preferably 55 cm,
wherein the cannula (CA107, CA108) is adapted to be inserted endovascularly, preferably jugular, through superior vena cava (VC), through right atrium (RA) and inferior vena cava (IVC) with blood drainage from at least one renal vein (rV1, rV2) or from both renal veins (rV1, rV2) and with blood delivery into the right atrium (RA).

7. Cannula (CA1 to CA7) according to claim 6, wherein the maximal outer diameter of the cannula (CA1 to CA7) is in the range of 25 F to 36 F or preferably in the range of 29 F to 33 F.

8. Cannula (CA1 to CA7) according to one of the claims 1 to 7, wherein the cannula (CA1 to CA7) comprises at least one valve for directing the fluid flows depending on the direction of the fluid flow in the proximal portion (PP1 to PP7), preferably a movable and/or pivotable valve, and/or
wherein the at least one valve is arranged at the at least one intermediate opening (IO1 to IO7).

9. Cannula (CA1 to CA7) according to claim 8, wherein the valve comprises one of the following elements:
b1) a curved plate-shaped member that is mounted pivotable around an axis that is arranged transversally to a longitudinal axis of the cannula (CA1 to CA7), wherein the curved member is mounted at the intermediate opening (IO1 to IO7),
b2) a curved plate-shaped member that is curved along a first curvature line and that comprises a deflector element that is curved along a second curvature line that extends within an angle of 80 to 100 degrees relative to the first curvature line, preferably with an angle of 90 degrees,
b3) a wedge shaped element, preferably comprising a first wedge shaped portion and a second wedge shaped portion, wherein preferably both wedge shaped portion point in opposite directions with regard to each other, and wherein the first wedge shaped portion has as smaller wedge angle compared to the wedge angle of the second wedge shaped portion, preferably at least 5 degrees smaller or at least 10 degrees smaller.

10. Cannula (CA1 to CA7) according to one of the claims 1 to 9, wherein the cannula is adapted to deliver blood with a flow rate within the range of 2.5 liter per minute to 4 liter per minute or within the range of 3 liter per minute to 3.5 liter per minute.

11. Cannula (CA1 to CA7) according to one of the claims 1 to 10, wherein the cannula (CA1 to CA7) comprises at least one expandable arrangement at the distal portion (DP1 to DP7), preferably a cage arrangement or a balloon.

12. Cannula (CA1 to CA7) according to one of the claims 1 to 10, wherein the cannula (CA1 to CA7) comprises at least one expandable arrangement at the intermediate portion (IP1 to IP7), preferably a cage arrangement or a balloon.

13. Cannula (CA1 to CA7) according to one of the claims 1 to 10, wherein the cannula (CA1 to CA7) comprises at least one first expandable arrangement at the distal portion (DP1 to DP7), preferably a first cage arrangement or a first balloon, and
wherein the cannula comprises at least one second expandable arrangement at the intermediate portion (IP1 to IP7), preferably a cage arrangement or a balloon.

14. Cannula (CA1 to CA7) according to one of the claims 1 to 13, wherein the wall thickness of the cannula (CA1 to CA7) is within the range of 0.1 mm to 0.5 mm, and/or
wherein the wall of the cannula (CA1 to CA7) is reinforced by wires (CA1 to CA7), especially by metal wires, or by plastic fibers or by glass fibers.

15. Cannula (CA1 to CA7) according to one of the claims 1 to 14, wherein the inner wall of the cannula comprises at least one structure that effects a rotation of the fluid flow within the cannula (CA1 to CA7), preferably at least one helically wound protrusion and/or recess.

16. Assembly (A3, A4, A6) for endovascular blood circuit support, comprising:
at least one cannula (CA1 to CA7) according to one of the claims 1 to 15,
at least one variable volume reservoir (MP3, MP4, MP6) that has an aspiration phase for drawing fluid into the variable volume reservoir (MP3, MP4, MP6) and that has an expulsion phase for pressing the fluid out of the variable volume reservoir (MP3, MP4, MP6) or a pump that may be controlled to drive a fluid flow within the cannula (CA1 to CA7) into two different directions,
wherein the cannula (CA1 to CA7) is coupled or is adapted to be coupled directly to the at least one variable volume reservoir (MP3, MP4, MP6) or to the pump or wherein the assembly comprises at least one coupling conduit that is coupled or that is adapted to be fluidically coupled between the at least one cannula (CA1 to CA7) and the at least one variable volume reservoir (MP3, MP4, MP6) or the pump.

17. Assembly according to claim 16, wherein the cannula (CA1 to CA7) and the variable volume reservoir (MP3, MP4, MP6) or the pump form separate devices that may be coupled with each other to form a fluid circuit.

18. Assembly (A3, A4, A6) according to one of the claims 16 or 17, wherein the variable volume reservoir (MP3, MP4, MP6) comprises at least one membrane (M), preferably a flat membrane (M) or a toroidal membrane.

19. Assembly (A3, A4, A6) according to one of the claims 16 to 19, wherein the variable volume reservoir (MP3, MP4, MP6) comprises two ports for blood transport, preferably at the same side of the membrane (M) or of a membrane (M).

20. Assembly (A3, A4, A6) according to one of the claims 16 to 18, wherein the variable volume reservoir (MP3, MP4, MP6) comprises only one port for blood transport that is connected with the cannula (CA1 to CA7).

21. Assembly (A3, A4, A6) according to one of the claims 16 to 20, comprising at least one oxygenator device (OXY3, OXY4, OXY6).

22. Assembly (A3, A4, A6) according to claim 21, wherein the oxygenator device (OXY3, OXY4, OXY6) is adapted to be inserted or is inserted fluidically within one secondary branch of a fluid circuit only, and wherein the fluid flow flows through the oxygenator device (OXY3, OXY4, OXY6) only in one direction (Dir3b, Dir4b, Dir6b).

23. Assembly (A3, A4, A6) according to claim 21, wherein the oxygenator (OXY3, OXY4, OXY6) is adapted to be inserted or is inserted into a main branch of a fluid circuit between the cannula (CA1 to CA7) and the variable volume reservoir (MP3, MP4, MP6),
and wherein the fluid flow flows through the oxygenator device (OXY3, OXY4, OXY6) in two directions.

24. Assembly (A3, A4, A6) according to one of the claims 16 to 23, wherein the variable volume reservoir (MP3, MP4, MP6) is adapted to be used with an IABP (Intra-Aortic Balloon Pump) console that is not part of the assembly (A3, A4, A6)
or wherein the assembly (A3, A4, A6) comprises a control unit that is able to control the variable volume reservoir (MP3, MP4, MP6) or the pump depending on the heartbeat of a heart and/or on the pulse beat of a pulse of a subject, wherein the beat is detected or measured by at least one sensor.

25. Assembly (A3, A4, A6) according to one of the claims 16 to 24, wherein the variable volume reservoir (MP3, MP4, MP6) has a maximal pump volume equal to or greater of 50 ml or equal to or greater of 60 ml, preferably within the range of 60 ml to 160 ml or most preferably within the range of 80 ml to 120 ml.

26. Method for endovascular blood circuit support,
inserting a cannula (CA1 to CA7, CA107, CA108, CA109a, CAI10a) endovascularly through a vessel of a blood circuit,
wherein the cannula (CA1 to CA7) comprises:
a proximal portion (PP1 to PP7),
a distal portion (DP1 to DP7) that comprises at least one distal opening (DO1 to DO7),
at least one lumen portion (LP) that extends from the at least one proximal portion (PP1 to PP6) to the at least one distal opening (DO1 to DO7), and
at least one intermediate portion (IP1 to IP6) that is arranged between the proximal portion (PP1 to PP6) and the distal portion (DP1 to DP7),
wherein the intermediate portion (IP1 to IP6) comprises at least one intermediate opening (IO1 to IO7), and
a) wherein the intermediate portion (IP1 to IP6) is configured such that more than 90 volume percent of the fluid flow are drained from the intermediate opening (IO1 to IO7) if a fluid flow within the proximal portion (PP1 to PP6) is directed proximally and such that more than 90 volume percent are delivered through the at least one distal opening (DO1 to DO7) if a fluid flow within the proximal portion (PP1 to PP6) is directed distally,
drawing blood mainly from the at least one intermediate opening (IO1 to IO7) during a drainage phase and delivering blood out of the at least one distal opening (DO1 to DO7) during a delivery phase, or
b) wherein the intermediate portion (IP109a, IP110a) is configured such that more than 90 volume percent of the fluid flow are drained from the at least one distal opening (DO109a, DO110a) if a fluid flow within the proximal portion (PP109a, PP110a) is directed proximally and such that more than 90 volume percent are delivered through the intermediate opening (I1009a, IO110a) if a fluid flow within the proximal portion (PP1 to PP6) is directed distally,
drawing blood mainly from the at least one distal opening (DO109a, DO110a) during a drainage phase and delivering blood out of the at least one intermediate opening (IO109a, IO110a) during a delivery phase.

27. Method according to claim 26,
wherein the distal portion (DP1, DP2) of the cannula (CA1, CA2) is inserted endovascularly, preferably jugularly, through vena cava (VC), right atrium (RA), atrial septum (AS), left atrium (LA), left ventricle (LV) at least to ascending aorta (aAO),
wherein blood is drained into the at least one intermediate opening (IO1) from the left atrium (LA) or wherein blood is drained into the at least one intermediate opening (IO2) from the left ventricle (LV) and wherein blood is delivered out of the at least one distal opening (IO1, IO2) into the aorta (AO).

28. Method according to claim 26,
wherein the distal portion (DP3) of the cannula (CA3) is inserted endovascularly, preferably jugularly, through vena cava (VC), right atrium (RA), atrial septum (AS), left atrium (LA), left ventricle (LV) at least to ascending aorta (aAO),
wherein blood is drained into the at least one intermediate opening (IO3) from the right atrium (RA) or from the vena cava (VC) and wherein blood is delivered out of the at least one distal opening (DO3) into the aorta (AO), preferably into the ascending aorta (aAO),
wherein the blood is oxygenated after it is drained in and before it is delivered out of the cannula (CA3), preferably by at least one extracorporeal oxygenator (OXY3).

29. Method according to claim 26,
wherein a) the distal portion (DP4) of the cannula (CA4) is inserted endovascularly, preferably jugularly, through vena cava (VC) and punctured from the vena cava (VC) directly to aorta (AO),
wherein blood is drained into the at least one intermediate opening (IO4) from the vena cava (VC) and
wherein blood is delivered out of the at least one distal opening (DO4) into the aorta (AO),
wherein the blood is oxygenated after it is drained in and before it is delivered out, preferably by at least one extracorporeal oxygenator device (OXY4), or
wherein b) the distal portion (DP4) of the cannula (CA4) is inserted endovascularly, preferably jugularly, through vena cava (VC), right atrium (RA) and punctured from the right atrium (RA) directly to aorta (AO),
wherein blood is drained into the at least one intermediate opening (IO4) from the vena cava (VC) or from the right atrium (RA) and wherein blood is delivered out of the at least one distal opening (DO4) into the aorta (AO),
wherein the blood is oxygenated after it is drained in and before it is delivered out, preferably by at least, or
wherein c) the distal portion of the cannula (CA10a) is inserted endovascularly, preferably jugularly, through vena cava (VC) and punctured directly from the vena cava (VC) to a pulmonary artery (PA), wherein blood is drained into the at least one intermediate opening (IO10a) from the vena cava (VC) and wherein blood is delivered out of the at least one distal opening into the pulmonary artery (PA), or wherein d) the distal portion (DP10) of the cannula (CA10) is inserted endovascularly, preferably jugularly, through vena cava (VC), right atrium (RA) and punctured from the right atrium (RA) directly to a pulmonary artery (PA),
wherein blood is drained into the at least one intermediate opening (IO10) from the vena cavy (VC) or from the right atrium (RA) and wherein blood is delivered out of the at least one distal opening (DO10) into the pulmonary artery (PA).

30. Method according to claim 26,
wherein the distal portion (DP5) of the cannula (CA5) is inserted endovascularly, preferably jugularly, through vena cava (VC), right atrium (RA), right ventricle (RV), ventricle septum (VS), left ventricle (LV) at least to ascending aorta (aAO),
wherein blood is drained into the at least one intermediate opening (IO5) from the left ventricle (LV) and wherein blood is delivered out of the at least one distal opening (IO5) into the aorta (AO).

31. Method according to claim 26,
wherein the distal portion (DP6) of the cannula (CA6) is inserted endovascularly, preferably jugularly, through vena cava (VC), right atrium (RA), right ventricle (RV), ventricle septum (VS), the ventricle (LV) at least to ascending aorta (aAO),
wherein blood is drained into the at least one intermediate opening (IO6) from the vena cava (VC) or from the right atrium (RA) or from the right ventricle (RV) and wherein blood is delivered out of the at least one distal opening (DO6) into the aorta (AO),
wherein the blood is oxygenated after it is drained in and before it is delivered out, preferably by at least one extracorporeal oxygenator device (OXY6).

32. Method according to claim 26,
wherein the distal portion (DP7) of the cannula (CA7) is inserted endovascularly, preferably jugularly, through vena cava (VC), right atrium (RA), right ventricle (RV) at least to main pulmonary artery (PA), wherein blood is drained into the at least one intermediate opening (IO7) from the vena cava (VC) or from the right atrium (RA) or from the right ventricle (RV) and wherein blood is delivered out of the at least one distal opening (DO7) into the pulmonary artery (PA).

33. Method according to one of the claims 26 to 32, comprising:
coupling the proximal portion (PP1 to PP11) of the cannula (CA1 to CA1 1) to a variable volume reservoir (MP3, MP4, MP6) that may perform the aspiration phase for drawing fluid into the reservoir (MP3, MP4, MP6) and that may perform the expulsion phase for pressing the fluid out of the reservoir (MP3, MP4, MP6) or to a pump.

34. Method according to claim 33, wherein a control unit is used that is able to control the variable volume reservoir (MP3, MP4, MP6) or the pump depending on the heartbeat of a heart (H) and/or on pulse beat of a subject, wherein the beat is detected or measured by at least one sensor.

35. Method according to claim 34, wherein the control unit controls the variable volume reservoir (MP3, MP4, MP6) or the pump such that every heartbeat, preferably every beat of the left ventricle (LV) blood is delivered into a body (IO0) of a subject.

36. Method according to claim 34, wherein the control unit controls the variable volume reservoir (MP3, MP4, MP6) or the pump such that every second heartbeat, preferably every second beat of the left ventricle (LV) blood is delivered into a body (IO0) of a subject.

37. Method according to one of the claims 26 to 36, wherein the cannula (CA1 to CA7) is inserted endovascularly, preferably jugular, through a septum of the heart (H).

38. Method according to claim 37, wherein the cannula (CA1 to CA7) is punctured through the atrial septum (AS) and/or inserted.

39. Method according to claim 37, wherein the cannula (CA1 to CA7) is punctured and/or inserted through the ventricle septum (VS).

40. Method according to one of the claims 26 to 36, wherein the cannula (CA1 to CA7) is inserted endovascularly, preferably jugular, through vena cava (VC) and wherein the cannula (CA1 to CA7) is punctured and/or inserted transcaval from the vena cava (VC) or from right atrium (RA) at least to aorta (AO) or into a pulmonary artery (PA).

41. Method according to one of the claims 26 to 40, wherein a maximal outer diameter of the cannula (CA1 to CA7) is in the range of 25 Fr to 36 Fr or, preferably, in the range of 29 Fr to 33 Fr.

42. Method according to one of the claims 26 to 41, wherein a cannula (CA1 to CA7) according to one of the claims 1 to 15 is used and/or wherein an assembly (A3, A4, A6) according to one of the claims 16 to 25 is used.

43. Method according to claim 26,
wherein the distal portion of the bidirectional cannula (CA107, CA108) is inserted endovascularly,
preferably jugularly, through superior vena cava (SVC), right atrium (RA) and inferior vena cava (IVC) at least to or to a location which has a distance to the junction of the renal veins (rV1, rV2) into the inferior vena cava (IVC) equal to 10 cm or less than 10 cm, equal to 5 cm or less than 5 cm or equal to 2.5 cm or less than 2.5 cm,
wherein blood is drained into the at least one distal opening (DO107, DO108) and wherein blood is delivered out of the at least one intermediate opening (IO107, IO108) into the right atrium (RA).

44. Method according to claim 43, wherein the cannula (CA107, CA108) is connected with only one membrane pump (MP7) or with at least two membrane pumps (MP8a, MP8b) which are preferably operated in a parallel operation mode.

45. Method according to claim 26,
wherein the cannula (CA109a, CA110a) is a bidirectional cannula (CA109a, CA110a) and wherein the bidirectional cannula (CA109a, CA110a) is inserted through the at least one vessel of the blood circuit within an outer cannula (CA109b, CA110b) which is arranged in the at least one vessel of the blood circuit,
wherein the outer cannula (CA109b, CA110b) comprises:
a proximal portion (PP109b, PP110b),
a distal portion (DP109b, DP110b1, DP110b2) that comprises at least one distal opening (DO109b, D110b1, DO110b2),
a lumen portion (LP) that extends from the proximal portion (PP109b, PP110b1, PP110b2) to the at least one distal opening (DO109b, D110b1, DO110b2), and
at least one intermediate portion (IPIO9b, IP110b) that is arranged between the proximal portion (PP109b, PP110b) and the distal portion (DPIO9b, DPIIObi, DP110b2),
wherein the intermediate portion (IPIO9b, IP110b) of the outer cannula (CAIO9b, CA110b) comprises at least one lateral intermediate opening (IO109b, IO110b) which is configured to allow passage of the distal portion (DP109a, DP110a) of the bidirectional cannula (CA1 to CA7, CA109a, CA110a).

46. Method according to claim 45,
wherein before inserting the bidirectional cannula (CA1 to CA7, CA109a, CA110a), the outer cannula (CA109b, CA110b) is inserted through the at least one vessel of the blood circuit,
wherein the bidirectional cannula (CA109a, CA110a) is inserted into the outer cannula (CA109b, CA110b) until the distal portion (DP109a, DP110a) of the bidirectional cannula (CA109a, CA110a) extends through the intermediate opening (IO109b, IO110b) of the outer cannula (CA109b, CA110b) and the intermediate opening (IO109a, IO110a) of the bidirectional cannula (CA109a, CA110a) is arranged within the intermediate portion (IP109b, IP110b) of the bidirectional cannula (CA109a, CA110a).

47. Method according to any one of the claims 45 or 46,
wherein the distal portion (DP109b) of the outer cannula is (CA109b) is inserted endovascularly, preferably jugularly, through superior vena cava (SVC), right atrium (RA), right ventricle (RV) at least to the pulmonary artery (PA),
wherein the distal portion (DP109a) of the bidirectional cannula (CA109a) is inserted into the right atrium (RA) or into the inferior vena cava (IVC),
wherein blood is drained into the at least one distal opening (DO109a) of the bidirectional cannula (CA109a) and wherein blood is delivered out of the at least one intermediate opening (IO109a) of the bidirectional cannula (CA109a) and further through the at least one distal opening (DO109b) of the outer cannula (CA109b).

48. Method according to any one of the claims 45 or 46,
wherein the distal portion (DP11Gb1, DP110b2) of the outer cannula is (CA110b) is inserted endovascularly, preferably jugularly, through the superior vena cava (SVC), the right atrium (RA) and the atrial septum (AS) to the left atrium (LA) of the heart (H) or to the left ventricle (LV) or at least to the left ventricle (LV),
wherein the distal portion (DP110a) of the bidirectional cannula (CA110a) is inserted into the right atrium (RA), through the right ventricle (RV) and at least to the pulmonary artery (PA),
wherein blood is drained into the at least one distal opening (DO110) of the bidirectional cannula (CA110a) and wherein blood is delivered out of the at least one intermediate opening (IO110a) of the bidirectional cannula (CA110a) and further through the at least one distal opening (DO110b) of the outer cannula (CA110b).

49. Method according to claim 48, wherein the drained blood is enriched with oxygen and/or depleted from carbon dioxide outside of the body of a patient before it is delivered out of the at least one intermediate opening (IO10 Ga) of the bidirectional cannula (CA110a).

50. Cannula system (CS, CA109a, CA109b; CA110a, CA110b), comprising:
a bidirectional cannula (CA109a, CA110a) according to one of the claims 1 to 15,
and an outer cannula (CA109b, CA110b),
wherein the outer cannula (CA109b, CA110b) comprises:
a proximal portion (PP109b, PP110b),
a distal portion (DP109b, DP110b1, DP110b2) that comprises at least one distal opening (DO109b, D110b1, DO110b2),
a lumen portion that extends from the proximal portion (PP109b, PP110b1, PP110b2) to the at least one distal opening (DO109b, D110b1, DO110b2), and
at least one intermediate portion (IP109b, IP110b) that is arranged between the proximal portion (PP109b, PP110b) and the distal portion (DP109b, DP110b1, DP110b2),
wherein the intermediate portion (IP109b, IP110b) of the outer cannula (CA109b, CA110b) comprises at least one lateral intermediate opening (IO109b, IO110b) which is configured to allow passage of the distal portion (DP109a, DP110a) of the bidirectional cannula (CA1 to CA7, CA109a, CA110a).

51. Cannula system (CA109a, CA109b; CA110a, CA110b) according to claim 50, wherein the bidirectional cannula (CA109a, CA110a) and the outer cannula (CA109b, CA110b) are configured such that when the bidirectional cannula (CA109a, CA110a) is inserted into the outer cannula (CA109b, CA110b), the distal portion (DP109a) of the bidirectional cannula (CA109a, CA110a) extends through the intermediate opening (IO109b, IO110b) of the outer cannula (CA109b, CA110b) and the intermediate opening (IO109a, IO110a) of the bidirectional cannula (CA109a, CA110a) is arranged within the intermediated portion (IP109b, IP110b) of the outer cannula (CA109b, CA110b) fluidly connected to the distal portion (DP109b, DP110b1, DP110b2) of the outer cannula (CA109b, CA110b).

52. Cannula system (CA109a, CA109b; CA110a, CA110b) according to claim 50 or 51, wherein the bidirectional cannula (CA109a, CA110a) and the outer cannula (CA109b, CA110b) are configured such that when the bidirectional cannula (CA109a, CA110a) is inserted into the outer cannula (CA109b, CA110b), a further lumen portion is defined between an outer surface of the bidirectional cannula (CA109a, CA110a) and an inner surface of the outer cannula (CA109b, CA110b) and wherein the further lumen portion is closed at its distal end and/or at its proximal end.

53. Cannula system (CA109a, CA109b; CA110a, CA110b) according to any one of the claims 50 to 52, wherein the outer diameter of the bidirectional cannula (CA109a, CA110a) is at most 4 French or at most 2 French smaller than the outer diameter of the outer cannula (CA109b, CA110b), preferably in a portion along the longitudinal axis of the bidirectional cannula (CAIO9a, CA110a) between the proximal portion (PP109a, PP110a) of the bidirectional cannula (CA109a, CA110a) and the intermediate portion (IP109a, IP110a) of the bidirectional cannula (CA109a, CA10a) when the bidirectional cannula (CA109a, CA110a) is inserted into the outer cannula (CA109b, CA110b).

54. Cannula system (CA109a, CA109b; CA110a, CA110b) according to any one of the claims 50 to 53, comprising a proximal valve (V9a, V10a), preferably a hemostasis valve, at the proximal portion (PP109b, PP110b) of the outer cannula (CA109b, CA110b), wherein preferably the proximal valve (V9a, V10a) is configured to allow insertion of the bidirectional cannula (CA109a, CA110a) through the proximal hemostatic valve (V9a, V10a) into the outer cannula (CA109b, CA110b).

55. Cannula system (CA109a, CA109b; CA110a, CA110b) according to any one of the claims 50 to 54, comprising an inner intermediate valve (V9b, V10b), preferably a hemostasis valve, at the intermediate portion (IP109b, IP110b) of the outer cannula (CA109b, CA110b), wherein preferably the intermediate valve (V9b, V10b) is configured to allow insertion of the bidirectional cannula (CA109a, CA110a) through the intermediate hemostatic valve (V9a, V10a).

56. Cannula system (CA109a, CA109b; CA110a, CA110b) according to any one of the claims 50 to 55, comprising a valve (V9c, V10c), preferably a hemostasis valve, at the intermediate opening (IO109b, IO110b) of the outer cannula (CA109b, CA110b), wherein preferably the valve (V9c, V10c) at the intermediate opening (IO109b, IO110b) is configured to allow passage of the distal portion (DP109a, DP110a) of the bidirectional cannula (CA109a, CA110a) through the intermediate hemostatic valve (V9c, V10c).

57. Cannula system (CA109a, CA109b; CA110a, CA110b) according to any one of the claims 50 to 56, wherein the outer cannula (CA109b, CA110b) comprises a kink (K) in the intermediate portion (IP109b, IP110b) of the outer cannula (CA109b, CA110b),
wherein preferably the kink (K) includes an angle in the range of 80 degrees to 130 degrees, preferably 110 degrees, and
wherein the intermediate opening (IO109b, IO110b) of the outer cannula (CA109b, CA110b) is arranged at the kink (K).

58. Cannula system (CAIO9a, CA109b; CA110a, CA110b) according to any one of the claims 50 to 57, wherein the cannula system (CA109a, CA109b; CA110a, CA110b) is adapted to be used for the method according to any one of the claims 45 to 49.

59. Assembly according to any one of the claims 16 to 25,
wherein at least one cannula (CA1 to CA7, CA109a, CA110a) is a bidirectional cannula (CA1 to CA7, CA109a, CA111a),
the assembly further comprising:
an outer cannula (CA109b, CA110b),
wherein bidirectional cannula (CA1 to CA7, CA109a, CA110a) is adapted to be inserted into the outer cannula (CA109b, CA110b),
wherein the outer cannula (CA109b, CA110b) comprises:
a proximal portion (PP109b, PP110b),
a distal portion (DP109b, DP110b1, DP110b2) that comprises at least one distal opening (DO109b, D110b1, DO110b2),
a lumen portion that extends from the proximal portion (PP109b, PP110b1, PP110b2) to the at least one distal opening (DO109b, D110b1, DO110b2), and
at least one intermediate portion (IP109b, IP110b) that is arranged between the proximal portion (PP109b, PP110b) and the distal portion (DP109b, DP110b1, DP110b2),
wherein the intermediate portion (IP109b, IP110b) of the outer cannula (CA109b, CA110b) comprises at least one intermediate opening (IO109b, IO110b) which is configured to allow passage of the distal portion (DP109a, DP110a) of the bidirectional cannula (CA1 to CA7, CA109a, CAI10a).

60. Assembly according to one of the claims 16 to 25 or to claim 59, comprising at least two variable volume reservoirs (MP8a, MP8b; MP9a, MP9b).

61. Cannula (CA109b, CA110b), preferably outer cannula (CA109b, CA110b) of a cannula system according to one of the claims 50 to 58 or outer cannula (CA109b, CA110b) of an assembly according to claim 59 or 60, comprising:
a proximal portion (PP109b, PP110b),
a distal portion (DP109b, DP110b1, DP110b2) that comprises at least one distal opening (DO09b, D110b1, DO110b2),
a lumen portion (LP) that extends from the proximal portion (PP109b, PP110b1, PP110b2) to the at least one distal opening (DO09b, D110b1, DO110b2), and
at least one intermediate portion (IP109b, IP110b) that is arranged between the proximal portion (PP109b, PP110b) and the distal portion (DP109b, DP110b1, DP110b2),
wherein the intermediate portion (IP109b, IP110b) of the outer cannula (CA109b, CA110b) comprises at least one lateral intermediate opening (IO109b, IO110b) which is configured to allow passage of the distal portion (DP109a, DP110a) of a further cannula, preferably of a the bidirectional cannula (CA1 to CA7, CA109a, CA10a).

62. Cannula (CA109b, CA110b) according to claim 61, comprising a kink (K) in the intermediate portion, wherein preferably the kink (K) includes an angle in the range of 80 degrees to 130 degrees, preferably 110 degrees, and
wherein the intermediate opening (IO109b, IO110b) of the outer cannula (CA109b, CA110b) is arranged at the kink (K).

63. Cannula (CA109b, CA110b) according to claim 62, wherein the intermediate portion (IP109b, IP110b) comprises a conical portion (ConP) or a decreasing diameter portion which reduces its outer diameter at positions which are more distally than other positions of the decreasing diameter portion,
wherein the portion varies in outer diameter by at least 3 French or by at last 4 French or by at least 5 French, preferably by less than 10 French or by less than 8 French.

64. Cannula (CA109b, CA110b) according to any one of the claims 61 to 63, wherein the conical portion (ConP) or the decreasing diameter portion is between the intermediate portion (IP109b, IP110b) of the cannula (CA109b, CA110b) and a distal portion (DP109b, DP110b) of the cannula (CA109b, CA110b), and
wherein the distal portion (DP109b, DP110b) comprises an essentially constant diameter portion comprising a length of at least 10 cm or of at least 20 cm, preferably less than 30 cm.

65. Cannula (CA109b, CA110b) according to any one of the claims 61 to 64, wherein the distal portion (DP109b, DP110b) comprises an outer diameter of at least 19 French, of at least 21 French, of at least 23 French, of at least 25 French, of at least 27 French or of at least 29 French, preferably of less than 33 French or less than 31 French.

66. Cannula (CA109b, CA110b) according to any one of the claims 61 to 65, wherein the distance between the proximal portion (PP109b, PP110b) and the intermediate opening (IO109b, IO110b) is at least 20 cm or at least 25 cm, preferably less than 35 cm or less than 30 cm.

67. Cannula (CA109b, CA110b) according to any one of the claims 61 to 66, wherein the cannula (CA109b, CA110b) is adapted to be used as an outer cannula (CA109b, CA110b) in a method according to any one of the claims 45 to 49.
